(19) United States
(12) Patent Application Publication
(10) Pub. No.: US 2021/0032304 A1
(43) Pub. Date: Feb. 4, 2021

(54) FIBROBLAST GROWTH FACTOR ANALOGS AND USES THEREOF

(71) Applicant: University of Florida Research Foundation, Inc., Gainesville, FL (US)

(72) Inventors: Paul Gerson Okunieff, Gainesville, FL (US); Steven G. Swarts, Archer, FL (US); Zhenhuan Zhang, Gainesville, FL (US); Steven Bingrong Zhang, Gainesville, FL (US)

(21) Appl. No.: 16/969,062

(22) PCT Filed: Feb. 13, 2019
(86) PCT No.: PCT/US2019/017790

§ 371 Date: Aug. 11, 2020

Related U.S. Application Data
(60) Provisional application No. 62/629,722, filed on Feb. 13, 2018.

(51) Int. Cl. C07K 14/50; A61P 39/00; A61K 38/00

(52) U.S. Cl. CPC C07K 14/50; A61K 38/00; A61P 39/00

(57) ABSTRACT

Disclosed herein are peptide analogs of human FGF. These peptide analogs exhibit improved therapeutic activity and fewer side effects when used in humans. Also disclosed are pharmaceutical or cosmetic compositions comprising the FGF analogs and pharmaceutically or cosmetically acceptable carriers or excipients. Also provided are methods of treating or preventing a disease in a subject that involves administering to the subject the disclosed FGF analogs.

### CROSS-REFERENCE TO RELATED APPLICATIONS

This application claims benefit of U.S. Provisional Application No. 62/629,722, filed Feb. 13, 2018, which is hereby incorporated herein by reference in its entirety.

### SEQUENCE LISTING

This application contains a sequence listing filed in electronic form as an ASCII.txt file entitled “222110-2730_5 T25” created on Feb. 9, 2019. The content of the sequence listing is incorporated herein in its entirety.

### BACKGROUND

While growth factors are used extensively to treat high-dose chemotoxicity, they have provided only minor benefits to patients suffering from radiotoxicity. The relative failure of agents such as G-CSF for GI mitigation is primarily due to the number of different tissues affected by radiation that are not covered by this marrow-specific growth factor. Studies of FGF-P, a multipotent growth factor peptide analog of mouse FGF, can greatly impact understanding of which non-hematopoietic diseases can benefit from growth factors. For example, FGF-P is currently being used preclinically. In those studies, FGF-P pretreatment produced improved survival and proliferation of pancreatic beta cells after transplantation in diabetic animals. FGF-P is also under development as an additive for an “after-sun” lotion to complement sunscreen protection.

In addition, FGF analogs can also be used in humans in improved healing in a variety of wound types (e.g., burns, skin grafts, ischemic ulcers, and donor sites for skin grafts), for treatment of human gingivitis, and myocardial stem cell transplantation in ischemic disease.

FGF-P is optimized for mice and not humans and thus, the doses and benefits of FGF-P in mice might be different from those observed in humans. Therefore, FGF analogs of human FGF are desirable.

### SUMMARY

Disclosed herein are FGF peptide analogs of human FGF. Compared to FGF-P, which is a mouse FGF analog, the analogs of human FGF exhibit improved therapeutic activity and fewer side effects when used in humans. FGF analogs of human FGF, and variants thereof, are hereinafter referred to as “FGF-PT” or “FGFPT.”

In certain embodiments, the FGF-PTs comprise or consist of the core sequence of SEQ ID NO: 2 or a variant of SEQ ID NO: 2 having one or more substitutions in the core sequence. In addition to one or more substitutions in the core sequence, the FGF-PTs can also be conjugated to one or more moieties. One or more amino acids from the core sequence or substituents of the core sequence can be conjugated to one or more moieties.

Also disclosed herein are pharmaceutical or cosmetic compositions comprising an FGF-PT and pharmaceutically or cosmetically acceptable carriers or excipients.

Methods of treating or preventing a disease in subject by administering to the subject a composition comprising FGF-PT are also provided. Methods of ameliorating adverse side effects of therapies, for example, radiotherapy or chemotherapy, are also provided.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1B show improved survival and weight recovery at different doses of FGF-P and FGF-PT after irradiation at 18 Gy. Improved survival was observed with FGF-P of 5-20 mg/kg. FIG. 1A. Survival. FIG. 1B. Body weight.

FIGS. 2A-2B show improved survival and weight recovery at different doses of FGF-P and FGF-PT after irradiation at 19.5 Gy. Improved survival was observed at all FGF-P, FGF-PT, and FGF2 doses. Recovery was observed after severe weight loss. FIG. 2A. Survival. FIG. 2B. Body weight.

FIGS. 3A-3B show improved survival and weight recovery at different doses of FGF-P and FGF-PT after irradiation at 20.5 Gy. Improved survival was observed at many FGF-P, FGF-PT, and FGF2 doses, particularly, with FGF-P doses of 5-20 mg/kg with recovery after very severe weight loss.

FIG. 4 shows survival rate (%) in 8-9 week old NIH Swiss male mice that have received a 18 Gy sub-TBI (one hind leg protected out of the radiation field) after subcutaneous doses of 10 and 20 mg/kg of FGF-PT administered once daily for three days beginning 24 hours post-irradiation, rhFGF2 administered at a dose of 0.3 mg/kg subcutaneously once at 24 hours post-irradiation, or vehicle control (normal saline) administered once daily for three days beginning 24 hours post irradiation.

FIG. 5 shows survival rate (%) in 8-9 week old NIH Swiss male mice that have received a 19 Gy sub-TBI (one hind leg protected out of the radiation field) after subcutaneous doses of 10 and 20 mg/kg of FGF-PT administered once daily for three days beginning 24 hours post-irradiation, rhFGF2 administered at a dose of 0.3 mg/kg subcutaneously once at 24 hours post-irradiation, or vehicle control (normal saline) administered once daily for three days beginning 24 hours post irradiation.

FIG. 6 illustrates a surface plasmon resonance method.

FIGS. 7A to 7C are fitted kinetic binding plots of hrFGF2 to the FGFR-2 alpha IIIc (FIG. 7A), FGFR-1 alpha IIIc (FIG. 7B), and FGFR-1 alpha IIIb (FIG. 7C) receptors. Five kinetic runs, one each for FGF-PT concentrations of 9.50, 39.0, 156 nM, are shown.

FIGS. 8A to 8C are kinetic binding plots of FGF-PT to the FGFR-2 alpha IIIc (FIG. 8A), FGFR-1 alpha IIIc (FIG. 8B), and FGFR-1 alpha IIIb (FIG. 8C) receptors. Five kinetic runs, one each for FGF-PT concentrations of 1.35, 2.70, 5.40, 10.8, and 21.6 uM, are shown.

### BRIEF DESCRIPTION OF THE SEQUENCES

SEQ ID NO: 1: Sequence of the conserved active site of mouse FGF2 (YRSRKYSSWYVALKR).

SEQ ID NO: 2: Sequence of the conserved active site of human FGF2 (YRSRKYTSWYVALKR).

SEQ ID NO: 3: Sequence of an FGF analog of mouse FGF2 (CYRSRKYSSWYVALKRC).

SEQ ID NO: 4: Sequence of an FGF analog of human FGF2 (CYRSRKYTSWYVALKRC).

SEQ ID NO: 5: The sequence of SEQ ID NO:2 with various substitutions.

### DETAILED DESCRIPTION

As used herein, the singular forms “a”, “an” and “the” are intended to include the plural forms as well, unless the context clearly indicates otherwise. Further, to the extent that the terms “including”, “includes”, “having”, “has”, “with” or variants thereof are used in either the detailed description and/or the claims, such terms are intended to be inclusive in a manner similar to the term “comprising”.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed. In the context of numerical parameters where the terms “about” is used, these parameters can be varied within a range of 0-10% around the stated value (X±10%).

In the present disclosure, ranges are stated in shorthand, so as to avoid having to set out at length and describe each and every value within the range. Any appropriate value within the range can be selected, where appropriate, as the upper value, lower value or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, such as for dose ranges, combinations and subcombinations of ranges (e.g., subranges within the disclosed range), specific embodiments therein are intended to be explicitly included.

“Pharmaceutically acceptable” means approved or approvable by a regulatory agency of the Federal or a state government or the corresponding agency in countries other than the United States or that is listed in the U.S. Pharmacopoeia or other generally recognized pharmacopoeia for use in animals, and more particularly, in humans.

“Pharmaceutically acceptable salt” refers to a salt of a peptide that is pharmaceutically acceptable and that possesses the desired pharmacological activity of the parent peptide. In particular, such salts are non-toxic may be inorganic or organic acid addition salts and base addition salts.

“Pharmaceutically acceptable vehicle” refers to a diluent, adjuvant, excipient or carrier with which a peptide is administered. A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

“Subject” includes humans or non-human animals, particularly, mammals, such as bovine, porcine, canine, rodent or feline animals. The terms “patient” and “subject” are used interchangeably herein.

“Treating” or “treatment” of any disease refers, in one embodiment, to ameliorating the disease (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease, either physically, (e.g., stabilization of a discernible symptom), physiologically, (e.g., stabilization of a physical parameter) or both. In yet another embodiment, “treating” or “treatment” refers to delaying the onset of the disease.

As used herein, the terms “reducing,” “inhibiting,” “blocking,” “preventing,” alleviating,” or “relieving” when referring to a peptide, mean that the peptide brings down the occurrence, severity, size, volume or associated symptoms of a disease, event or activity by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90% or 100% compared to how the disease, event or activity would normally exist without application of the peptide or a composition comprising the peptide. The terms “increasing,” “elevating,” “enhancing,” “upregulating,” “improving,” or “activating” when referring to a peptide mean that the peptide increases the occurrence or activity of a disease, event or activity by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, 100%, 150%, 200%, 250%, 300%, 400%, 500%, 750% or 1000% compared to how the disease, event or activity would normally exist without application of the peptide or a composition comprising the peptide.

In treatment methods, a therapeutically effective amount of a pharmaceutical agent is administered to a subject suffering from or diagnosed as having such a disease. A “therapeutically effective amount” means an amount or dose sufficient to generally bring about the desired therapeutic or prophylactic benefit in patients in need of such treatment for the designated disease.

Effective amounts or doses of the disclosed peptides can be ascertained by routine methods such as modeling, dose escalation studies or clinical trials, and by taking into consideration routine factors, e.g., the mode or route of administration or drug delivery, the pharmacokinetics of the peptide, the severity and course of the disease, the subject's previous or ongoing therapy, the subject's health status and response to drugs, and the judgment of the treating physician. An example of a dose is in the range of from about 0.001 to about 200 mg of peptide per kg of subject's body weight per day, preferably about 0.05 to 100 mg/kg/day or about 1 to 35 mg/kg/day, in single or divided dosage units (e.g., BID, TID, QID). For a 70-kg human, an illustrative range for a suitable dosage amount is from about 0.05 to about 7 g/day or about 0.2 to about 2.5 g/day.

“Amino acid” as used herein refers to organic compounds containing amine and carboxyl functional groups, along with a side chain specific to each amino acid. Amino acids for use in the disclosed peptides include natural or synthetic amino acids.

“Peptides,” and equivalent expressions, refer to refer to compounds having a molecular structure comprising chiefly or entirely of a number of amino acids bonded together via peptide bonds.

Analogs of FGF, such as FGF-P, are effective against acute gastrointestinal syndrome. Administration of FGF analogs in mice induce proliferation of stem cells in the crypt regions; improved recovery of bone marrow cellularity after total body irradiation (TBI) leading reduced endotoxemia; improved day 7 and overall survival; reduced physiologically responses including bleeding, fluid loss, diarrhea, and weight loss; and improved cytokine expression.

Few experimental agents satisfactorily mitigate acute radiation toxicity and none are approved for human use. FGF-P provides some beneficial effects in mice; however, because FGF-P is a mouse FGF analog, it may not be as effective in humans.

Therefore, disclosed herein are analogs of human FGF. The sequence of the active site of the mouse FGF2 (SEQ ID NO: 1) differs from the sequence of the active site of the human FGF2 (SEQ ID NO: 2) at position 7. The human FGF2 has threonine instead of serine in the mouse FGF2. Therefore, analogs of human FGF are referred to herein as FGF-PT.

Accordingly, disclosed herein is an FGF-PT peptide, which refers to a peptide having the sequence of SEQ ID NO: 2 or a variant of SEQ ID NO: 2, said variant having at least one amino acid substitution at position X^{y}, where y is selected from 1, 2, 3, 4, 5, 6, 7, 8, 9, 10, 11, 12, 13, 14 or 15. The amino acid positions in the SEQ ID NOs disclosed herein are referenced as X^{y}, wherein X represents the amino acid and y represents the position of the amino acid in the amino acid sequence. For SEQ ID NO: 2, X¹=Y, X²=R, X³=S, X⁴=R, X⁵=K, X⁶=Y, X⁷=T, X⁸=S, X⁹=W, X¹⁰=Y, X¹¹=V, X¹²=A, X¹³=L, X¹⁴=K, and X¹⁵=R.

X¹ of SEQ ID NO: 2 can be substituted with an analog of tyrosine. Such analogs include D-tyrosine, tryptophan, phenylalanine, 4-amino phenylalanine, 4-chloro phenylalanine, 4-bromo phenylalanine, 4-iodo phenylalanine, 4-fluoro phenylalanine, 4-trifluoromethyl phenylalanine, 4-guanidino phenylalanine, 4-aminomethyl phenylalanine, 4-phosphonomethyl phenylalanine, 3,4-dichloro phenylalanine, 3,4-difluoro phenylalanine, 3,4-dihydroxy phenylalanine, 4-benzoyl phenylalanine, 4-bis(2-chloroethyl)amino phenylalanine, 3-nitro tyrosine, 3-chloro tyrosine, 3-amino tyrosine, 3-fluorotyrosine or 2,6-dimethyl-tyrosine. The tyrosine analog can be in L or D configuration. Additional analogs of tyrosine are known to a skilled artisan and can be used in the disclosed peptides.

X² of SEQ ID NO: 2 can be substituted with an analog of arginine. Such analogs include D-arginine, lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline ornithine, diamino butyric acid, diamino propionic acid. The arginine analog can be in L or D configuration. Additional analogs of arginine are known to a skilled and can be used in the disclosed peptides.

X³ of SEQ ID NO: 2 can be substituted with an analog of serine. Such analogs include D-serine, threonine, alanine, 2-Amino-4-hydroxybutyric acid (homoserine), O-benzyl homoserine, O-methyl homoserine, 3-amino-2-hydroxypropanoic acid, O-phosphoserine, O-sulfoserine, O-nitroserine, β-(2-Thienyl)-serine, O-phosphothreonine, O-sulfothreonine, 0-nitrothreonine. The serine analog can be in L or D configuration. Additional analogs of serine are known to a skilled artisan and can be used in the disclosed peptides.

X⁴ of SEQ ID NO: 2 can be substituted with an analog of arginine. Such analogs include D-arginine, lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid. The arginine analog can be in L or D configuration. Additional analogs of arginine are known to a skilled artisan and can be used in the disclosed peptides.

X⁵ of SEQ ID NO: 2 can be substituted with an analog of lysine. Such analogs include arginine, D-lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid. The lysine analog can be in L or D configuration. Additional analogs of lysine are known to a skilled artisan and can be used in the disclosed peptides.

X⁶ of SEQ ID NO: 2 can be substituted with an analog of tyrosine. Such analogs include D-tyrosine, tryptophan, phenylalanine, 4-amino phenylalanine, 4-chloro phenylalanine, 4-bromo phenylalanine, 4-iodo phenylalanine, 4-fluoro phenylalanine, 4-trifluoromethyl phenylalanine, 4-guanidino phenylalanine, 4-aminomethyl phenylalanine, 4-phosphonomethyl phenylalanine, 3,4-dichloro phenylalanine, 3,4-difluoro phenylalanine, 3,4-dihydroxy phenylalanine, 4-benzoyl phenylalanine, 4-bis(2-chloroethyl)amino phenylalanine, 3-nitro tyrosine, 3-chloro tyrosine, 3-amino tyrosine, 3-fluoro tyrosine or 2,6-dimethyl-tyrosine. The tyrosine analog can be in L or D configuration. Additional analogs of tyrosine are known to a skilled artisan and can be used in the disclosed peptides.

X⁷ of SEQ ID NO: 2 can be substituted with an analog of threonine. Such analogs include D-threonine, serine, β-hydroxy norvaline or O-phospho threonine. The threonine analog can be in L or D configuration. Additional analogs of threonine are known to a skilled artisan and can be used in the disclosed peptides.

X⁸ of SEQ ID NO: 2 can be substituted with an analog of serine. Such analogs include D-serine, threonine, alanine, 2-Amino-4-hydroxybutyric acid (homoserine), O-benzyl homoserine, O-methyl homoserine, 3-amino-2-hydroxypropanoic acid, 0-phosphoserine, O-sulfoserine, O-nitroserine, β-(2-Thienyl)-serine, O-phosphothreonine, O-sulfothreonine, O-nitrothreonine. The serine analog can be in L or D configuration. Additional analogs of serine are known to a skilled artisan and can be used in the disclosed peptides.

X⁹ of SEQ ID NO: 2 can be substituted with an analog of tryptophan. Such analogs include D-tryptophan, tyrosine, phenylalanine, 1-methyl tryptophan, 5-fluoro tryptophan, 4-methyl tryptophan, 6-fluoro tryptophan, 7-azatryptophan, 5-benzyloxy tryptophan, 5-bromo tryptophan, 5-chloro tryptophan, 5-hydroxy tryptophan, 5-methoxy tryptophan, 6-chrloro tryptophan, 6-methyl tryptophan, 7-methyl tryptophan, or 7-azatryptophan. The tryptophan analog can be in L or D configuration. Additional analogs of tryptophan are known to a skilled artisan and can be used in the disclosed peptides.

X¹⁰ of SEQ ID NO: 2 can be substituted with an analog of tyrosine. Such analogs include D-tyrosine, tryptophan, phenylalanine, 4-amino phenylalanine, 4-chloro phenylalanine, 4-bromo phenylalanine, 4-iodo phenylalanine, 4-fluoro phenylalanine, 4-trifluoromethyl phenylalanine, 4-guanidino phenylalanine, 4-aminomethyl phenylalanine, 4-phosphonomethyl phenylalanine, 3,4-dichloro phenylalanine, 3,4-difluoro phenylalanine, 3,4-dihydroxy phenylalanine, 4-benzoyl phenylalanine, 4-bis(2-chloroethyl)amino phenylalanine, 3-nitro tyrosine, 3-chloro tyrosine, 3-amino tyrosine, 3-fluoro tyrosine or 2,6-dimethyl-tyrosine. The tyrosine analog can be in L or D configuration. Additional analogs of tyrosine are known to a skilled artisan and can be used in the disclosed peptides.

X¹¹ of SEQ ID NO: 2 can be substituted with an analog of valine. Such analogs include D-valine, isoleucine, leucine, β-cyclohexyl alanine, α,β-diamino propionic acid, propargylglycine, α-amino isobutyric acid, β-(2-pyridyl) alanine, β-(3-pyridyl) alanine, β-cyclopropyl alanine, β-t-butyl alanine, β-allyloxy carbonyl-α,β-diaminopropionic acid, 4-fluoro phenylglycine, 5,5,5-trifluoro leucine, 2-amino-3-(dimethylamino)-propionic acid or 2-amino caproic acid, allylglycine. The valine analog can be in L or D configuration. Additional analogs of valine are known to a skilled artisan and can be used in the disclosed peptides.

X¹² of SEQ ID NO: 2 can be substituted with an analog of alanine. Such analogs include D-alanine, serine, β-cyclohexyl alanine, α,β-diamino propionic acid, propargylglycine, α-amino isobutyric acid, β-(2-pyridyl) alanine, β-(3-pyridyl) alanine, β-cyclopropyl alanine, β-t-butyl alanine, β-allyloxy carbonyl-α,β-diaminopropionic acid, 4-fluoro phenylglycine, 5,5,5-trifluoro leucine, 2-amino-3-(dimethylamino)-propionic acid or 2-amino caproic acid, allylglycine. The alanine analog can be in L or D configuration. Additional analogs of valine are known to a skilled artisan and can be used in the disclosed peptides.

X¹³ of SEQ ID NO: 2 can be substituted with an analog of leucine. Such analogs include D-leucine, isoleucine, valine, β-cyclohexyl alanine, α,β-diamino propionic acid, propargylglycine, α-amino isobutyric acid, β-(2-pyridyl) alanine, β-(3-pyridyl) alanine, β-cyclopropyl alanine, β-t-butyl alanine, β-allyloxy carbonyl-α,β-diaminopropionic acid, 4-fluoro phenylglycine, 5,5,5-trifluoro leucine, 2-amino-3-(dimethylamino)-propionic acid or 2-amino caproic acid, allylglycine. The leucine analog can be in L or D configuration. Additional analogs of leucine are known to a skilled artisan and can be used in the disclosed peptides.

X¹⁴ of SEQ ID NO: 2 can be substituted with an analog of lysine. Such analogs include arginine, D-lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid. The lysine analog can be in L or D configuration. Additional analogs of lysine are known to a skilled artisan and can be used in the disclosed peptides.

X¹⁵ of SEQ ID NO: 2 can be substituted with an analog of arginine. Such analogs include D-arginine, lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid. The arginine analog can be in L or D configuration. Additional analogs of arginine are known to a skilled artisan and can be used in the disclosed peptides.

In some embodiments, one or more amino acids from X¹ to X¹⁵ of SEQ ID NO: 2 are each substituted with an analog of the corresponding amino acids. For example, one, two, three, four, five, six, seven, eight, nine, ten, eleven, twelve, thirteen, fourteen or fifteen amino acids from SEQ ID NO: 2 can be substituted with the analogs of the corresponding amino acids. Any combinations of one to fifteen substitutions over first to fifteenth positions of SEQ ID NO: 2 are envisioned. Therefore, in some embodiments, the peptide has the amino acid sequence SEQ ID NO:5, which is the combination of each of the above subsitutions.

In further embodiments, an amino acid is added to the C-terminus and/or N-terminus of SEQ ID NO: 2. In the case of cysteine, cysteine or an analog of cysteine is added to the C and/or the N terminus, The analog of cysteine can either be capable of forming disulfide bonds, such as penicillamine or homocysteine, or incapable of forming a disulfide linkage, such as buthionine, α-methyl methionine, selenomethionine, S-[2-(4-pyridyl)ethyl] cysteine, S-diphenylmethyl cysteine, S-trityl-homocysteine, S-trityl penicillamine. The analog of cysteine can be in L or D configuration.

An amino acid can be added to only the C-terminus, only the N-terminus or both the C and the N termini of SEQ ID NO: 2. In some embodiments, a first analog of cysteine is added to the C-terminus of SEQ ID NO: 2 and a second analog of cysteine is added to the N-terminus. When analogs of cysteine are added to both the C and the N termini of SEQ ID NO: 2, any combinations of the analogs listed above and the analogs known in the art are envisioned.

A structural variant of the FGF-PT is the cyclization of the peptide. One embodiment of a cyclic FGF-PT is a disulfide linkage between the amino and carboxyl termini. The linkage occurs through oxidation of the thiol functional groups on both cysteines added to the C and the N termini of SEQ ID NO: 2. In other embodiments of a disulfide linked cyclized FGF-PT include disulfide bonds between cysteine and a cysteine analog, or between cysteine analogs, added to the amino and carboxyl termini of SEQ ID NO: 2. The cysteine analogs include pencillamine and homocysteine and analogs known in the art. These embodiments can be envisioned as a disulfide linkage formed between an analog of cysteine at either the C or N-termini, and a cysteine added to the opposite terminus, or between analogs of cysteine when added to both the C and the N termini, or between any combinations of cysteine analogs when added both the C and N termini of SEQ ID NO: 2.

In another embodiment of a cyclized FGF-PT is the N-to-C (or head-to-tail) cyclization through amide bond formation between amino and carboxyl termini. In one embodiment is a amide bond between X¹ to X¹⁵ of SEQ ID NO: 2. In other embodiments, the natural amino acids and analogs of these amino acid known to the art are added to either the C or N termini, or both termini of SEQ ID NO: 2 and an amide bond formed between the resulting amino acids at the C terminus and N terminus

In addition to one or more substitutions in the core sequence and/or additional of amino acids at the C and/or the N termini of SEQ ID NO: 2, the FGF-PTs can also be conjugated to one or more moieties. One or more amino acids from the core sequence, substituents of the core sequence, or those added at the C and/or the N-termini can be conjugated to one or more moieties, including poly(ethylene)glycol (PEG) chains of varying lengths and long chain (C-10-C18) fatty acids and fatty amines.

Also disclosed are salts of the peptides described herein. The salts can be with an inorganic acid, such as hydrochloric acid, hydrobromic acid, perchloric acid, nitric acid, thiocyanic acid, sulfuric acid, and phosphoric acid; an organic acid, such as trifluoroacetic acid (TFA), formic acid, acetic acid, propionic acid, glycolic acid, lactic acid, pyruvic acid, oxalic acid, malonic acid, succinic acid, maleic acid, and fumaric acid; or a salt with a base, such as sodium hydroxide, ammonium hydroxide, potassium hydroxide, and organic bases such as mono-, di-, trialkyl and aryl amines, and substituted ethanolamines.

Further salts include: (1) acid addition salts, formed with organic acids such as hexanoic acid, cyclopentanepropionic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-di sulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent peptide either is replaced by a metal ion, e.g., an alkali metal ion, an alkaline earth ion or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the peptide contains a basic functionality, salts of non-toxic organic or inorganic acids, such as hydrochloride, hydrobromide, tartrate, mesylate, acetate, maleate, oxalate and the like.

Certain embodiments provide amorphous forms of salts of the peptides disclosed herein. Such amorphous forms are advantageous for oral, pulmonary, buccal, suppository delivery.

Pharmaceutical Compositions and Routes of Administration

Disclosed herein are pharmaceutical compositions comprising an FGF-PT and a pharmaceutically acceptable carrier and/or an excipient. Pharmaceutical compositions can be suitable for administration via intramuscular, subcutaneous, intrathecal, intravenous or intraperitoneal by infusion or injection. Pharmaceutical compositions of an FGF-PT can be prepared in water, optionally mixed with a nontoxic surfactant. Under ordinary conditions of storage and use, these pharmaceutical compositions can contain a preservative to prevent the growth of microorganisms. Pharmaceutical compositions can comprise, in addition to an FGF-PT, a sugar and/or a poly-alcohol excipients. The pH of the pharmaceutical compositions can be between 4 to 10, preferably, between 5 to 9, even more preferably between 6 to 8, and particularly, about 7.

The pharmaceutical dosage forms suitable for injection or infusion can include sterile aqueous solutions or dispersions or sterile powders comprising the peptide that are adapted for the extemporaneous preparation of sterile injectable or infusible solutions or dispersions, optionally encapsulated in liposomes. Preferably, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained by, for example, the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the peptide in the required amount in the appropriate solvent as described herein with various of the other ingredients enumerated herein, as required, preferably followed by sterilization, for example, filter sterilization. In the case of sterile powders for the preparation of sterile injectable solutions, the preferred methods of preparation are vacuum drying and freeze drying techniques, which yield a powder of an FGF-PT plus any additional desired ingredient present in the previously sterile-filtered solutions.

The disclosed compositions can also be administered orally, in combination with a pharmaceutically acceptable vehicle such as an inert diluent or an assimilable edible carrier. They may be enclosed in hard or soft shell gelatin capsules, may be compressed into tablets or may be incorporated directly with the food of the patient's diet.

For oral therapeutic administration, an FGF-PT may be combined with one or more excipients and used in the form of ingestible tablets, buccal tablets, troches, capsules, elixirs, suspensions, syrups, wafers, and the like. Such compositions and preparations can contain at least 0.1% of an FGF-PT. The percentage of an FGF-PT present in such compositions and preparations may, of course, be varied and can conveniently be between about 2% to about 60% of the weight of a given unit dosage form. The amount of an FGF-PT in such therapeutically useful compositions is such that an effective dosage level will be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid, and the like; a lubricant such as magnesium stearate; and a sweetening agent such as sucrose, fructose, lactose or aspartame, or a flavoring agent such as peppermint, oil of wintergreen or cherry flavoring may be added.

When the unit dosage form is a capsule, it may contain, in addition to materials of the above type, a liquid carrier, such as a vegetable oil or a polyethylene glycol.

Various other materials may be present as coatings or for otherwise modifying the physical form of the solid unit dosage form. For instance, tablets, pills or capsules may be coated with gelatin, wax, shellac or sugar, and the like. A syrup or elixir may contain an FGF-PT, sucrose or fructose as a sweetening agent, methyl and propylparabens as preservatives, a dye, and flavoring such as cherry or orange flavor.

Of course, any material used in preparing any unit dosage form should be pharmaceutically acceptable and substantially non-toxic in the amounts employed.

In addition, an FGF-PT may be incorporated into sustained-release preparations and devices. For example, an FGF-PT may be incorporated into time release capsules, time release tablets, time release pills, and time release polymers or nanoparticles.

Pharmaceutical compositions for topical administration of an FGF-PT to the epidermis (mucosal or cutaneous surfaces) can be formulated as ointments, creams, lotions, gels or as a transdermal patch. Such transdermal patches can contain penetration enhancers such as linalool, carvacrol, thymol, citral, menthol, t-anethole, and the like. Ointments and creams can, for example, include an aqueous or oily base with the addition of suitable thickening agents, gelling agents, colorants, and the like. Lotions and creams can include an aqueous or oily base and typically also contain one or more emulsifying agents, stabilizing agents, dispersing agents, suspending agents, thickening agents, coloring agents, and the like. Gels preferably include an aqueous carrier base and include a gelling agent such as cross-linked polyacrylic acid polymer, a derivatized polysaccharide (e.g., carboxymethyl cellulose), and the like.

Pharmaceutical compositions suitable for topical administration in the mouth (e.g., buccal or sublingual administration) include lozenges comprising the composition in a flavored base, such as sucrose, acacia or tragacanth; pastilles comprising the composition in an inert base such as gelatin and glycerin or sucrose and acacia; and mouthwashes comprising the peptide in a suitable liquid carrier. The pharmaceutical compositions for topical administration in the mouth can include penetration enhancing agents, if desired.

Useful solid carriers include finely divided solids such as talc, clay, microcrystalline cellulose, silica, alumina, and the like. Other solid carriers include nontoxic polymeric nanoparticles or microparticles. Useful liquid carriers include water, alcohols or glycols, or water/alcohol/glycol blends, in which an FGF-PT can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings or sprayed onto the affected area using pump-type or aerosol sprayers.

Thickeners such as synthetic peptides, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver an FGF-PT to the skin are known in the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), all of which are hereby incorporated by reference.

The concentration of an FGF-PT in such formulations can vary widely depending on the nature of the formulation and intended route of administration. For example, the concentration of an FGF-PT in a liquid composition, such as a lotion, can preferably be from about 0.1-25% by weight, or, more preferably, from about 0.5-10% by weight. The concentration in a semi-solid or solid composition such as a gel or a powder can preferably be about 0.1-5% by weight, or, more preferably, about 0.5-2.5% by weight.

Pharmaceutical compositions for spinal administration or injection into amniotic fluid can be provided in unit dose form in ampoules, pre-filled syringes, small volume infusion or in multi-dose containers, and can include an added preservative. The compositions for parenteral administration can be suspensions, solutions or emulsions, and can contain excipients such as suspending agents, stabilizing agents, and dispersing agents.

A pharmaceutical composition suitable for rectal administration comprises an FGF-PT in combination with a solid or semisolid (e.g., cream or paste) carrier or vehicle. For example, such rectal compositions can be provided as unit dose suppositories. Suitable carriers or vehicles include cocoa butter and other materials commonly used in the art.

According to some embodiments, pharmaceutical compositions suitable for vaginal administration are provided as pessaries, tampons, creams, gels, pastes, foams or sprays containing a peptide disclosed herein in combination with carriers as are known in the art. Alternatively, compositions suitable for vaginal administration can be delivered in a liquid or solid dosage form.

Also disclosed herein are pharmaceutical compositions suitable for intra-nasal administration. Such intra-nasal compositions comprise an FGF-PT in a vehicle and suitable administration device to deliver a liquid spray, dispersible powder or drops. Drops may be formulated with an aqueous or non-aqueous base also comprising one or more dispersing to agents, solubilizing agents or suspending agents. Liquid sprays are conveniently delivered from a pressurized pack, an insufflator, a nebulizer or other convenient means of delivering an aerosol comprising the peptide. Pressurized packs comprise a suitable propellant such as dichlorodifluoromethane, trichlorofluoromethane, dichlorotetrafluoroethane, carbon dioxide or other suitable gas as is well known in the art. Aerosol dosages can be controlled by providing a valve to deliver a metered amount of an FGF-PT.

The peptide can be combined with an inert powdered carrier and inhaled by the subject or insufflated.

Pharmaceutical compositions for administration by inhalation or insufflation can be provided in the form of a dry powder composition, for example, a powder mix of an FGF-PT and a suitable powder base such as lactose or starch. Such powder composition can be provided in unit dosage form, for example, in capsules, cartridges, gelatin packs or blister packs, from which the powder can be administered with the aid of an inhalator or insufflator.

The exact amount (effective dose) of an FGF-PT will vary from subject to subject, depending on, for example, the species, age, weight, and general or clinical disease of the subject, the severity or mechanism of any disease being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either in cell culture assays or in suitable animal models. The animal model may also be used to determine the appropriate concentration ranges and routes of administration. Such information can then be used to determine useful doses and routes for administration in humans. Methods for the extrapolation of effective dosages in mice and other animals to humans are known to the art; for example, see U.S. Pat. No. 4,938,949, which is hereby incorporated by reference. A therapeutic dose can also be selected by analogy to dosages for comparable therapeutic agents.

The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months or even years.

In general, however, a suitable dose will be in the range of from about 0.001 to about 100 mg/kg of body weight per day, preferably from about 0.01 to about 100 mg/kg of body weight per day, more preferably, from about 0.1 to about 50 mg/kg of body weight per day or even more preferred, in a range of from about 1 to about 10 mg/kg of body weight per day. For example, a suitable dose may be about 1 mg/kg, 10 mg/kg or 50 mg/kg of body weight per day.

An FGF-PT can be conveniently administered in unit dosage form, containing for example, about 0.05 to about 10000 mg, about 0.5 to about 10000 mg, about 5 to about 1000 mg or about 50 to about 500 mg of peptide per unit dosage form.

An FGF-PT can be administered to achieve peak plasma concentrations of, for example, from about 0.25 to about 200 μM, about 0.5 to about 75 μM, about 1 to about 50 μM, about 2 to about 30 μM or about 5 to about 25 μM. Exemplary desirable plasma concentrations include at least 0.25, 0.5, 1, 5, 10, 25, 50, 75, 100 or 200 μM. For example, plasma levels may be from about 1 to about 100 micromolar or from about 10 to about 25 micromolar. This may be achieved, for example, by the intravenous injection of a 0.05 to 5% solution of an FGF-PT, optionally in saline or orally administered as a bolus containing about 1 to about 100 mg of an FGF-PT. Desirable blood levels may be maintained by continuous or intermittent infusion.

An FGF-PT can be included in the compositions within a therapeutically useful and effective concentration range, as determined by routine methods that are well known in the medical and pharmaceutical arts. For example, a typical composition can include one or more of FGF-PT at a concentration in the range of at least about 1 mg/ml, preferably at least about 4 mg/ml, more preferably at least 5 mg/ml and most preferably at least 6 mg/ml.

An FGF-PT may conveniently be presented in a single dose or as divided doses administered at appropriate intervals, for example, as one dose per day or as two, three, four or more sub-doses per day. The sub-dose itself may be further divided, e.g., into a number of discrete loosely spaced administrations; such as multiple inhalations from an insufflator.

Optionally, the pharmaceutical compositions can include one or more other therapeutic agents, e.g., as a combination therapy. The additional therapeutic agent(s) will be included in the compositions within a therapeutically useful and effective concentration range, as determined by routine methods that are well known in the medical and pharmaceutical arts. The concentration of any particular additional therapeutic agent may be in the same range as is typical for use of that agent as a monotherapy or the concentration may be lower than a typical monotherapy concentration if there is a synergy when combined with a peptide disclosed herein.

Methods of Treatment

Disclosed herein is a method of preventing or treating a disease affecting rapidly proliferating tissue comprising administering to a subject an effective amount of an FGF-PT provided herein, thereby preventing or treating a disease affecting rapidly proliferating tissue or one or more symptoms thereof. The disease can be caused by an insult to the rapidly proliferating tissue. The insult can be radiation exposure, exposure to a chemical agent or a microorganism or a combination thereof.

Also disclosed is a method of treating leukopenia (e.g., neutropenia) of a subject exposed to an insult affecting rapidly proliferating tissues (such as radiation, chemotherapy, and chemical/biological warfare agents with radiomimetic properties) comprising administering to the subject a therapeutically effective amount of an FGF-PT provided herein.

Disclosed herein is a method of protecting and/or regenerating gastrointestinal tissues of a subject exposed to an insult affecting rapidly proliferating tissues (such as radiation, chemotherapy, and chemical/biological warfare agents with radiomimetic properties) comprising administering to the subject a therapeutically effective amount of an FGF-PT.

Disclosed herein are methods of preventing and/or treating a disease (e.g., alimentary mucositis, bone marrow failure, radiation induced prostatitis, virginitis and/or urethritis, a disease of hematopoiesis or a cardiovascular/central nervous system syndrome) or ameliorating a symptom (e.g., diarrhea, skin burn, sores, fatigue, dehydration, inflammation, hair loss, ulceration of oral mucosa, xerostomia, and bleeding) associated with an insult affecting rapidly proliferating tissues (such as radiation, chemotherapy, and chemical/biological warfare agents with radiomimetic properties) comprising administering to a subject in need thereof a prophylactically or therapeutically effective amount of a peptide comprising an FGF-PT.

In one example, an FGF-PT is administered to a subject prior to the subject's exposure to the insult. In another embodiment, an FGF-PT is administered to a subject after the subject's exposure to the insult, but prior to any disease associated with the insult or a symptom thereof developed in the subject. In another embodiment, an FGF-PT is administered to a subject after one or more diseases associated with the insult or symptoms thereof developed in the subject. In another embodiment, an FGF-PT is administered to a subject in need thereof both prior to the development of any radiation associated disease and/or symptom (e.g., prior to the occurrence of the insult, and/or after the occurrence of the insult but prior to the development of any disease) and after the development of a radiation associated disease. In yet another embodiment, an FGF-PT is administered to a subject who is at risk of exposing to an insult affecting rapidly proliferating tissues (such as radiation, chemotherapy, and chemical/biological warfare agents with radiomimetic properties).

In a specific example, an FGF-PT is administered to a subject in need thereof no more than 24 hours, 20 hours, 15 hours, 10 hours or 5 hours prior to the subject's exposure to an insult affecting rapidly proliferating tissues (e.g., radiation, chemotherapy, and chemical/biological warfare agents with radiomimetic properties). In another embodiment, an FGF-PT is administered to a subject in need thereof 3 days, 2 days, 1 day prior to exposure to radiation (day −3, −2, and −1), the day exposed to radiation (day 0), and the day after exposure to the radiation (day 1), respectively. In yet another embodiment, an FGF-PT is administered to a subject in need thereof on day −1, 0, and 1, respectively. Many more dosing schedules can be used, and such schedules are encompassed by the disclosed methods.

Disclosed herein is a method of improving survival of subjects exposed to an insult affecting rapidly proliferating tissues (such as radiation, chemotherapy, and chemical/biological warfare agents with radiomimetic properties) comprising administering to the subjects a prophylactically or therapeutically effective amount of an FGF-PT. The therapeutically effective dose may be a single dose, two doses or more than two doses of a composition.

A single prophylactic dose of an FGF-PT can be administered to a subject followed by an insult affecting rapidly proliferating tissues (such as radiation, chemotherapy, and chemical/biological warfare agents with radiomimetic properties), where such prophylactic dose causes a defined, short acting proliferative effect on various compartments in the proliferating tissues (e.g., intestinal villi). In another embodiment, more than a single prophylactic dose, which may be two or more than two doses of an FGF-PT is administered to a subject exposed to an insult affecting rapidly proliferating tissues (such as radiation, chemotherapy, and chemical/biological warfare agents with radiomimetic properties) to preventing, treating or ameliorating a symptom associated with the insult.

An insult affecting rapidly proliferating tissues can be radiation exposure. In some embodiments, an insult affecting rapidly proliferating tissues is one or more alkylating agents, one or more vesicant agents (e.g., mustard agents) or one or more other chemotherapeutic agents or a combination thereof. In some embodiments, an insult affecting rapidly proliferating tissues is a radiation exposure in combination with one or more alkylating agents, one or more mustard agents or one or more other chemotherapeutic agents.

An FGF-PT can be used in combination with one or more other therapies known in the art to prevent, treat or ameliorate one or more symptoms associated with an insult affecting rapidly proliferating tissues (such as radiation, chemotherapy, and chemical/biological warfare agents with radiomimetic properties).

Examples of diseases that can be treated by the compositions disclosed herein include, but are not limited to, alimentary mucositis, oral mucositis, gastrointestinal mucositis, hematopoiesis, anemia, leukopenia, thrombocytopenia, pancytopenia or a clotting disease. The disease can also be bone marrow failure, graft-versus-host disease, radiation induced prostatitis, virginitis, urethritis or a cardiovascular/central nervous system syndrome.

When the subject has been exposed to radiation, the radiation exposure can result in diarrhea, skin burn, sores, fatigue, dehydration, inflammation, hair loss, ulceration of alimentary tract mucosa, xerostomia, bleeding or a combination thereof.

Further disclosed herein is a method of promoting angiogenesis in a subject in need thereof, the method comprising administering to the subject an effective amount of an FGF-PT, thereby promoting angiogenesis in the subject.

An FGF-PT may be used for any disease for which inducing angiogenesis provides a therapeutic or palliative effect. Angiogenesis includes inducing vascularized tissue growth and physiological blood vessel formation. One particular application is angiogenesis at sites of ischemia, such as in the heart or a limb, to improve local or regional blood flow. In general, FGF-PT can be employed to limit, repair or reverse damage to ischemic tissues, both internal and external. Thus, FGF-PT can be employed for use in treatment of various intractable ulcers, including deep wound ulcers. Examples include bed sores, such as pressure-induced decubitus ulcers, ulcerative extremities, gangrenous extremities, diabetic ulcers and the like. Internal ulcers include oral mucosa ulcers, gastrointestinal ulcers, such as gastric ulcers, duodenal ulcers or ulcers associated with trauma or other injury. FGF-P can also be used to restore aspects of epithelial integrity in diseases including those characterized by inflammation of the gastrointestinal tract, including diseases such as inflammatory bowel disease, ulcerative colitis and Crohn's disease.

FGF-PT can thus be employed generally for wound healing, including surgically-induced, disease-induced and trauma-induced wounds. An FGF-PT disclosed herein can be employed to assist in healing of muscles, skin, bone, cartilage and other tissues of the body. In surgical procedures, the peptide can be employed to limit, prevent or treat abdominal wall incisional hernias or to reduce fascial wound failure. In some embodiments, the composition provides sustained release of the peptides.

Injuries of the bone, which may be traumatic injuries and also include injuries resulting from diseases and degenerative diseases that may be treated by an FGF-PT include fractures, open fractures, compound fractures, non-union fractures, segmental bone filling, boney voids, ischemic osteonecrosis, including avascular necrosis, and the like. An FGF-PT can also be employed in various orthopedic procedures, including procedures in which any device or fixture is intended to be fixed to bone or any disease for which osteoinduction is desired. Thus, FGF-PT can be employed for spinal fixation procedures using cages, rods, and other implants. FGF-PT can be employed for other forms of spinal fusion and treatment of vertebral fractures and degenerative discs. FGF-PT can be employed for joint replacement procedures, including but not limited to application as a coating component on joint prostheses. An FGF-PT can be employed for distraction osteogenesis and similar procedures for lengthening or otherwise altering bone. An FGF-PT can also be employed in dental applications.

Injuries of the dermis may be treated by an FGF-PT disclosed herein, such as chemical, radiation or heat induced burns.

In another aspect, an FGF-PT can be employed in treatment of various cardiovascular diseases. In one aspect, therapeutic angiogenesis induced by an FGF-PT can serve to salvage chronically ischemic myocardium. In another aspect, FGF-PT can serve to increase cardiac resistance to injury and to guard against secondary injury after an acute ischemic insult, such as at the time of reperfusion. Thus depending on the disease state and the clinical objectives, the FGF-PT can be administered either acutely or chronically. In another aspect, an FGF-PT can serve to treat or ameliorate arterial occlusion.

In another aspect, an FGF-PT can be used to improve pulmonary function in patients with emphysema and other chronic obstructive pulmonary diseases. For pulmonary applications, an FGF-PT can be delivered as an aerosol of microparticles or may be administered by intratracheal means, such as using controlled-release microspheres.

In another aspect, FGF-PT can be used for treatment or improvement of neurological deficits including in the treatment of Huntington's disease, Parkinson's disease or Alzheimer's disease, and the like or after occlusive cerebrovascular disease. An FGF-PT can be used in combination with other agents, including specifically bone marrow stromal cell transplantation.

An FGF-PT can have particular application in patients with compromised or reduced immune systems, and patients with diseases, such as diabetes, in which chronic or ischemic ulcers, wounds and the like are more common or in accelerating tissue transplants in such patients.

An FGF-PT can be useful as a biologically active agent for coating of medical devices, such as for instance, sutures, implants and medical instruments to promote biological responses, for instance, to stimulate growth and proliferation of cells or healing of wounds. FGF-PT can also be used in treatment of bone fracture healing, bone void fillers, aneurysm healing and treatment, preparation of artificial organ implant sites and in therapeutic angiogenesis.

During bone repair and insufficient or interrupted angiogenic response following injury inhibits osseous regeneration and is also thought to contribute to the pathophysiology of fibrous union, osteomyelitis, and osteoradionecrosis. The importance of angiogenesis in bone repair is reinforced by the ability of anti-angiogenic compounds to inhibit ectopic bone formation and by the ability of an FGF-PT to accelerate bone healing.

The term “medical device” as used herein means a device that has one or more surfaces in contact with an organ, tissue, blood or other bodily fluid in an organism, preferably a mammal, particularly, a human. Medical devices include, for example, extracorporeal devices for use in surgery such as blood oxygenators, blood pumps, blood sensors, tubing used to carry blood, and the like which contact blood that is returned to the patient. The term can also include endoprostheses implanted in blood contact in a human or animal body, such as vascular grafts, stents, pacemaker leads, heart valves, and the like that are implanted in blood vessels or in the heart. The term can further include devices for temporary intravascular use such as catheters, guide wires, and the like that are placed in blood vessels or the heart for purposes of monitoring or repair. The term can further include nerve electrodes, muscle electrodes, implantable pulse generators, implantable drug pumps, and defibrillators. Moreover, the term medical device can include sutures, graft materials, wound coverings, nerve guides, bone wax, aneurysm coils, embolization particles, microbeads, dental implants, bone prostheses, tissue scaffolds, artificial joints or controlled release drug delivery devices.

The surface of the medical device can be formed from any of the commonly used materials suitable for use in medical devices, such as for instance, stainless steel, titanium, platinum, tungsten, ceramics, polyurethane, polytetrafluoroethylene, extended polytetrafluoroethylene, polycarbonate, polyester, polypropylene, polyethylene, polystyrene, polyvinyl chloride, polyamide, polyacrylate, polyurethane, polyvinyl alcohol, polycaprolactone, polylactide, polyglycolide, polysiloxanes (such as 2,4,6,8-tetramethylcyclotetrasiloxane), natural rubbers or artificial rubbers, or block peptide or copeptide thereof.

Methods for coating biological molecules onto the surfaces of medical devices are known. See for instance U.S. Pat. No. 5,866,113 to Hendriks et al., the specification of which is hereby incorporated by reference. Tsang et al. in U.S. Pat. No. 5,955,588 teach a non-thrombogenic coating composition and methods for using the same on medical devices, and is incorporated herein by reference. Zamora et al. in U.S. Pat. No. 6,342,591 teach an amphipathic coating for medical devices for modulating cellular adhesion composition, and is incorporated herein by reference.

An FGF-PT can be delivered to a mammal, the method including (i) providing a medical device coated on its surface with FGF-PT, the peptide being bound to the surface of the medical device by non-covalent bonds; and (ii) placing the medical device onto a surface of or implanting the medical device into, the mammal.

The medical device can be an aneurysm coil or other vaso-occlusive device, and an FGF-PT serves to induce endothelial cell attachment, proliferation and/or migration, and optionally further angiogenesis, such that a permanent and substantial blockage of the blood vessel into which the aneurysm coil or other vaso-occlusive device is placed results. Aneurysm coils and vaso-occlusive devices are described in U.S. Pat. Nos. 6,866,155, 6,835,185, 6,656,218, 6,656,201, 6,638,291, 6,616,617, 6,551,305, 6,416,541, 6,383,204, 6,306,153, 6,221,066, 6,171,326, 6,168,615, 6,165,194, 6,159,165, 6,136,015 and 6,102,932, incorporated here by reference as if set forth in full.

Disclosed herein are methods of preventing and/or treating diseases of epithelial cells and/or mesenchymal cells comprising administering an FGF-PT to a subject in need thereof. Also disclosed are methods of stimulating proliferation, differentiation or migration of epithelial cells and/or mesenchymal cells, comprising administering an FGF-PT to a subject in need thereof.

Epithelial membranes are continuous sheets of cells with contiguous cell borders that have characteristic specialized sites of close contact called cell junction. Such membranes, which can be one or more cells thick, contain no capillaries. Epithelia are attached to the underlying connective tissue by a component known as a basement membrane, which is a layer of intercellular material of complex composition that is distributed as a thin layer between the epithelium and the connective tissue.

Stratified squamous non-keratinizing epithelium is common on wet surfaces that are subject to considerable wear and tear at sites where absorptive function is not required. The secretions necessary to keep such surfaces wet have to come from appropriately situated glands. Sites lined by this type of epithelium include the esophagus and the floor and sides of the oral cavity.

Simple columnar epithelium is made up of a single layer of tall cells that again fit together in a hexagonal pattern. In simple secretory columnar epithelium, the columnar cells are all specialized to secret mucus in addition to being protective. Sites of this type of epithelium is present include the lining of the stomach.

A simple columnar epithelium that is made up of absorptive cells as well as secretory cells lines the intestine. To facilitate absorption, this membrane is only one cell thick. Interspersed with cells that are specialized for absorption, there are many goblet cells that secrete protective mucus.

Mesenchymal cells are stem cells that can differentiate into, e.g., osteoblasts, chondrocytes, myocytes, and adipocytes. Mesenchymal-epithelial interactions play an important role in the physiology and pathology of epithelial tissues. Mesenchymal cells may associate with epithelium basement membrane (e.g., pericytes and perivascular monocyte-derived cells (MDCs)) or reside within epithelium (MDCs and T cells). The nature of the interactions between mesenchymal cells and tissue-specific cells may depend on the tissue type (e.g., brain versus epidermis) or on the prevention or allowance/stimulation of differentiation of cells into the suicidal state (apoptosis) by mesenchymal cells in a given epithelium. Specialized mesenchymal cells, such as pericytes, MDCs, and T lymphocytes, may significantly influence the differentiation and aging of epithelial cells.

The stromal compartment of the cavities of bone is composed of a net-like structure of interconnected mesenchymal cells. Stromal cells are closely associated with bone cortex, bone trabecule and to the hemopoietic cells. The bone marrow-stromal microenvironment is a complex of cells, extracellular matrix (ECM) with growth factors and cytokines that regulate osteogenesis and hemopoiesis locally throughout the life of the individual. The role of the marrow stroma in creating the microenvironment for bone physiology and hemopoiesis lies in a specific subpopulation of the stroma cells. They differentiate from a common stem cell to the specific lineage each of which has a different role. Their combined function results in orchestration of a 3-D-architecture that maintains the active bone marrow within the bone.

In adults, blood cells are produced by the bone marrow, the spongy material filling the body's bones. The bone marrow produces two blood cell groups, myeloid and lymphoid. The myeloid cell line includes, e.g., the following: (1) Immature cells called erythrocytes that later develop into red blood cells; (2) Blood clotting agents (platelets); (3) Some white blood cells, including macrophages (which act as scavengers for foreign particles), eosinophils (which trigger allergies and also defend against parasites), and neutrophils (the main defenders against bacterial infections). The lymphoid cell line includes, e.g., the lymphocytes, which are the body's primary infection fighters. Among other vital functions, certain lymphocytes are responsible for producing antibodies, factors that can target and attack specific foreign agents (antigens). Lymphocytes develop in the thymus gland or bone marrow and are therefore categorized as either B-cells (bone marrow-derived cells) or T-cells (thymus gland-derived cells).

Disclosed herein are methods of stimulating hematopoietic stem cell proliferation, comprising administering to a subject an FGF-PT, thereby stimulating hematopoietic stem cell proliferation.

Also disclosed are methods of optimizing hematopoietic stem cell engraftment, comprising administering to a subject an FGF-PT, thereby optimizing hematopoietic stem cell engraftment.

Further disclosed are methods of stimulating gastrointestinal stem cell proliferation, comprising administering to a subject an FGF-PT, thereby stimulating gastrointestinal stem cell proliferation.

Disclosed are methods to stimulate growth and proliferation of cells in a vertebrate animal, comprising administering to a vertebrate subject in need of such treatment an effective amount of an FGF-PT, thereby stimulating growth and proliferation of cells in a vertebrate animal. The cells can be crypt cells. The cells can be in the gastrointestinal tract.

Further disclosed are methods for treating an aneurysm in a vertebrate animal, comprising introducing an embolus generating vaso-occlusive device into the aneurysm, wherein the vaso-occlusive device comprises an effective amount of an FGF-PT, thereby treating the aneurysm in a vertebrate animal.

Further disclosed is a vaso-occlusive device, comprising an effective amount of a composition that augments fibroblast growth factor activity, which composition comprises an FGF-PT.

Further disclosed are methods to treat ulcerative colitis in a vertebrate animal, comprising administering to a vertebrate subject in need of such treatment an effective amount of an FGF-PT, thereby treating ulcerative colitis in a vertebrate animal.

According to the methods disclosed herein, an FGF-PT may be administered to a subject intramuscularly, subcutaneously, intrathecally, intravenously or intraperitoneally by infusion or injection.

All patents, patent applications, provisional applications, and publications referred to or cited herein are incorporated by reference in their entirety, including all figures and tables, to the extent they are not inconsistent with the explicit teachings of this specification.

The following are example aspects of the invention.

Aspect 1. A peptide having the sequence of SEQ ID NO: 2, provided that: a) one, two or, three amino acids of SEQ ID NO: 2 is substituted; b) an amino acid is added at the C-terminus and/or N-terminus of SEQ ID NO: 2; or c) one, two or, three amino acids of SEQ ID NO: 2 is substituted and an amino acid is added at the C-terminus and/or N-terminus of SEQ ID NO: 2.

Aspect 2. The peptide of aspect 1, wherein X¹ of SEQ ID NO: 2 is substituted with D-tyrosine, tryptophan, phenylalanine, 4-amino phenylalanine, 4-chloro phenylalanine, 4-bromo phenylalanine, 4-iodo phenylalanine, 4-fluoro phenylalanine, 4-trifluoromethyl phenylalanine, 4-guanidino phenylalanine, 4-aminomethyl phenylalanine, 4-phosphonomethyl phenylalanine, 3,4-dichloro phenylalanine, 3,4-difluoro phenylalanine, 3,4-dihydroxy phenylalanine, 4-benzoyl phenylalanine, 4-bis(2-chloroethyl)amino phenylalanine, 3-nitro tyrosine, 3-chloro tyrosine, 3-amino tyrosine, 3-fluoro tyrosine or 2,6-dimethyl-tyrosine.

Aspect 3. The peptide of aspect 1 or 2, wherein X² of SEQ ID NO: 2 is substituted with D-arginine, lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid.

Aspect 4. The peptide of any one of aspects 1 to 3, wherein X³ of SEQ ID NO: 2 is substituted with D-serine, threonine, alanine, 2-Amino-4-hydroxybutyric acid (homoserine), O-benzyl homoserine, O-methyl homoserine, 3-amino-2-hydroxypropanoic acid, O-phosphoserine, O-sulfoserine, O-nitroserine, β-(2-Thienyl)-serine, O-phosphothreonine, O-sulfothreonine, O-nitrothreonine.

Aspect 5. The peptide of any one of aspects 1 to 4, wherein X⁴ of SEQ ID NO: 2 is substituted with D-arginine, lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid.

Aspect 6. The peptide of any one of aspects 1 to 5, wherein X⁵ of SEQ ID NO: 2 is substituted with arginine, D-lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid.

Aspect 7. The peptide of any one of aspects 1 to 6, wherein X⁶ of SEQ ID NO: 2 is substituted with D-tyrosine, tryptophan, phenylalanine, 4-amino phenylalanine, 4-chloro phenylalanine, 4-bromo phenylalanine, 4-iodo phenylalanine, 4-fluoro phenylalanine, 4-trifluoromethyl phenylalanine, 4-guanidino phenylalanine, 4-aminomethyl phenylalanine, 4-phosphonomethyl phenylalanine, 3,4-dichloro phenylalanine, 3,4-difluoro phenylalanine, 3,4-dihydroxy phenylalanine, 4-benzoyl phenylalanine, 4-bis(2-chloroethyl)amino phenylalanine, 3-nitro tyrosine, 3-chloro tyrosine, 3-amino tyrosine, 3-fluoro tyrosine or 2,6-dimethyl-tyrosine.

Aspect 8. The peptide of any one of aspects 1 to 7, wherein X⁷ of SEQ ID NO: 2 is substituted with D-threonine, serine, β-hydroxy norvaline or O-phosphothreonine.

Aspect 9. The peptide of any one of aspects 1 to 8, wherein X⁸ of SEQ ID NO: 2 is substituted with D-serine, threonine, alanine, 2-Amino-4-hydroxybutyric acid (homoserine), O-benzyl homoserine, O-methyl homoserine, 3-amino-2-hydroxypropanoic acid, 0-phosphoserine, O-sulfoserine, O-nitroserine, β-(2-Thienyl)-serine, O-phosphothreonine, O-sulfothreonine, O-nitrothreonine.

Aspect 10. The peptide of any one of aspects 1 to 9, wherein X⁹ of SEQ ID NO: 2 is substituted with D-tryptophan, tyrosine, phenylalanine, 1-methyl tryptophan, 5-fluoro tryptophan, 4-methyl tryptophan, 6-fluoro tryptophan, 7-azatryptophan, 5-benzyloxy tryptophan, 5-bromo tryptophan, 5-chloro tryptophan, 5-hydroxy tryptophan, 5-methoxy tryptophan, 6-chrloro tryptophan, 6-methyl tryptophan, 7-methyl tryptophan or 7-azatryptophan.

Aspect 11. The peptide of any one of aspects 1 to 10, wherein X¹⁰ of SEQ ID NO: 2 is substituted with D-tyrosine, tryptophan, phenylalanine, 4-amino phenylalanine, 4-chloro phenylalanine, 4-bromo phenylalanine, 4-iodo phenylalanine, 4-fluoro phenylalanine, 4-trifluoromethyl phenylalanine, 4-guanidino phenylalanine, 4-aminomethyl phenylalanine, 4-phosphonomethyl phenylalanine, 3,4-dichloro phenylalanine, 3,4-difluoro phenylalanine, 3,4-dihydroxy phenylalanine, 4-benzoyl phenylalanine, 4-bis(2-chloroethyl)amino phenylalanine, 3-nitro tyrosine, 3-chloro tyrosine, 3-amino tyrosine, 3-fluoro tyrosine or 2,6-dimethyl-tyrosine.

Aspect 12. The peptide of any one of aspects 1 to 11, wherein X¹¹ of SEQ ID NO: 2 is substituted with D-valine, isoleucine, leucine, β-cyclohexyl alanine, α,β-diamino propionic acid, propargylglycine, α-amino isobutyric acid, β-(2-pyridyl) alanine, β-(3-pyridyl) alanine, β-cyclopropyl alanine, β-t-butyl alanine, β-allyloxy carbonyl-α,β-diaminopropionic acid, 4-fluoro phenylglycine, 5,5,5-trifluoro leucine, 2-amino-3-(dimethylamino)-propionic acid or 2-amino caproic acid, allylglycine.

Aspect 13. The peptide of any one of aspects 1 to 2, wherein X¹² of SEQ ID NO: 2 is substituted with D-alanine, serine, β-cyclohexyl alanine, α,β-diamino propionic acid, propargylglycine, α-amino isobutyric acid, β-(2-pyridyl) alanine, β-(3-pyridyl) alanine, β-cyclopropyl alanine, β-t-butyl alanine, β-allyloxy carbonyl-α,β-diaminopropionic acid, 4-fluoro phenylglycine, 5,5,5-trifluoro leucine, 2-amino-3-(dimethylamino)-propionic acid or 2-amino caproic acid, allylglycine.

Aspect 14. The peptide of any one of aspects 1 to 13, wherein X¹³ of SEQ ID NO: 2 is substituted with D-leucine, isoleucine, valine, β-cyclohexyl alanine, α,β-diamino propionic acid, propargylglycine, α-amino isobutyric acid, β-(2-pyridyl) alanine, β-(3-pyridyl) alanine, β-cyclopropyl alanine, β-t-butyl alanine, β-allyloxy carbonyl-α,β-diaminopropionic acid, 4-fluoro phenylglycine, 5,5,5-trifluoro leucine, 2-amino-3-(dimethylamino)-propionic acid or 2-amino caproic acid, allylglycine.

Aspect 15. The peptide of any one of aspects 1 to 14, wherein X¹⁴ of SEQ ID NO: 2 is substituted with arginine, D-lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid.

Aspect 16. The peptide of any one of aspects 1 to 15, wherein X¹⁵ of SEQ ID NO: 2 is substituted with D-arginine, lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid.

Aspect 17. The peptide of any one of aspects 1 to 16, wherein the substituted amino acid is in D-configuration.

Aspect 18. The peptide of any one of aspects 1 to 17, wherein one amino acid from X¹ to X¹⁵ of SEQ ID NO: 2 is substituted.

Aspect 19. The peptide of any one of aspects 1 to 18, wherein the peptide comprises the amino acid sequence SEQ ID NO: 5.

Aspect 20. The peptide of any one of aspects 1 to 19, wherein the peptide is cyclized by forming an amide bond between X¹ to X¹⁵ of SEQ ID NO: 2.

Aspect 21. The peptide of any one of aspects 1 to 20, wherein an amino acid is added at the C-terminus and/or N-terminus of SEQ ID NO: 2.

Aspect 22. The peptide of aspect 21, wherein the amino acid added to the C and the N terminus is cysteine, a combination of cysteine and an analog of cysteine, an analog of cysteine, or combination of analogs of cysteine that is capable of cyclizing the peptide through formation of a disulfide linkage.

Aspect 23. The peptide of aspect 21, wherein the amino acid added to the C and/or the N terminus is an analog of cysteine that is not capable of forming a disulfide linkage.

Aspect 24. The peptide aspect claim 21 or 23, wherein the amino acid is added only to the C-terminus or only to the N-terminus of SEQ ID NO: 2.

Aspect 25. The peptide of aspect 21, 22 or 23, wherein the amino acid is added to both the C and the N termini of SEQ ID NO: 2.

Aspect 26. The peptide of aspect 21, 22, 23, or 25, wherein a first amino acid is added to the C-terminus and a second amino acid is added to the N-terminus.

Aspect 27. The peptide of any one of aspects 1 to 26, wherein an amino acid is added at the C-terminus and/or N-terminus of SEQ ID NO: 2 is cyclized through formation of an amide bond between the resulting C-terminus and N-terminus.

Aspect 28. A salt of a peptide of any one of aspects 1 to 27.

Aspect 29. The salt of aspect 28, wherein the salt is with: hydrochloric acid, hydrobromic acid, perchloric acid, nitric acid, thiocyanic acid, sulfuric acid, phosphoric acid, trifluoroacetic acid (TFA), formic acid, acetic acid, propionic acid, hexanoic acid, cyclopentanepropionic acid, glycolic acid, lactic acid, pyruvic acid, oxalic acid, malic acid, malonic acid, succinic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-di sulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, sodium hydroxide, ammonium hydroxide, potassium hydroxide, mono-, di-, trialkyl and aryl amines, ethanolamines, diethanolamine, triethanolamine, N-methylglucamine, sodium, potassium, calcium, magnesium, ammonium, tetraalkylammonium.

Aspect 30. A composition comprising a peptide of any one of aspects 1 to 27 or a salt of aspect 28 or 29, and a pharmaceutically acceptable carrier and/or an excipient.

Aspect 31. A method of treating a disease in a subject, comprising administering to the subject a peptide of any one of aspects 1 to 27, a salt of aspect 28 or 29 or a composition of aspect 30.

Aspect 32. The method of aspect 31, wherein the disease affects rapidly proliferating tissue.

Aspect 33. The method of aspect 31 or 32, wherein the disease occurs due to radiation exposure.

Aspect 34. The method of aspect 32 or 33, wherein the disease is alimentary mucositis, oral mucositis, gastrointestinal mucositis, disease of hematopoiesis, anemia, leukopenia, thrombocytopenia, pancytopenia, clotting disease, bone marrow failure, graft-versus-host disease, radiation induced prostatitis, vaginitis, urethritis or a cardiovascular/central nervous system syndrome, diarrhea, skin burn, sores, fatigue, dehydration, inflammation, hair loss, ulceration of alimentary tract mucosa, xerostomia, bleeding, aneurysm or a combination thereof.

Aspect 35. The method of aspect 33 or 34, comprising administering the peptide to the subject: before exposed to radiation, after exposure to radiation and prior to the development of the disease or after exposure to radiation and after the development of the disease.

Aspect 36. The method of any of aspects 31 to 34, comprising inducing angiogenesis in a tissue in the subject by administering the peptide to the tissue, wherein inducing angiogenesis in the tissue treats the disease.

Aspect 37. The method of aspect 36, wherein the tissue is epidermis, eye, skin, urogenital tract, gastro-intestinal tract, cardiovascular tissue, muscle, connective tissue or neural tissue.

Aspect 38. The method of any of aspects 31 to 34, comprising stimulating growth and proliferation of cells in a tissue in the subject by administering the peptide to the tissue, wherein inducing growth and proliferation of cells treats the disease.

Aspect 39. The method of aspect 38, wherein the tissue is epidermis, eye, skin, urogenital tract, gastro-intestinal tract, cardiovascular tissue, muscle, connective tissue or neural tissue.

Following are examples which illustrate procedures for practicing the invention. These examples should not be construed as limiting. All percentages are by weight and all solvent mixture proportions are by volume unless otherwise noted.

### EXAMPLES

### Example 1: Efficacy of FGF-PT in Alleviating Radiation Damage

150 outbred stock (NIS) Swiss males (8-11 weeks) were subjected to 5 sub-TBI doses (0, 18, 19.5, 20.5, 21.5 Gy). Three single dosages of FGF-P were administered 24 hr post-IR (5.0, 10, and 20 mg/kg), and two single dosages of FGF-PT were administered 24 hr post-IR (10 and 20 mg/kg). One dosage of FGF2 (0.3 mg/kg) and a saline vehicle control was also administered. Animals were followed for signs of distress, survivors, and weight loss/gain for 30 days. Survivors at the end of 30 days were euthanized.

Radiation doses were chosen to bracket the highest dose with few or no deaths (LD₂₀) and the lowest dose with few or no survivors (LD₈₀), thus ensuring a range that includes the LD₅₀ and statistically delivers the highest accuracy and precision per animal.

TABLE 1

| subTBI | | FGF-P | FGF-P | FGF-P | FGF-PT | FGF-PT | FGF2 |
|---|---|---|---|---|---|---|---|
| Dose, | Sa- | 5.0 | 10 | 20 | 10 | 20 | 0.3 |
| Gy | line | mg/kg | mg/kg | mg/kg | mg/kg | mg/kg | mg/kg |
| 0 | 5 | 5 | 5 | 5 | 5 | 5 | 5 |
| 18 | 5 | 5 | 5 | 5 | 5 | 5 | 5 |
| 19.5 | 5 | 5 | 5 | 5 | 5 | 5 | 5 |
| 20.5 | 5 | 5 | 5 | 5 | 5 | 5 | 5 |
| 21.5 | 5 | 5 | 5 | 5 | 5 | 5 | 5 |

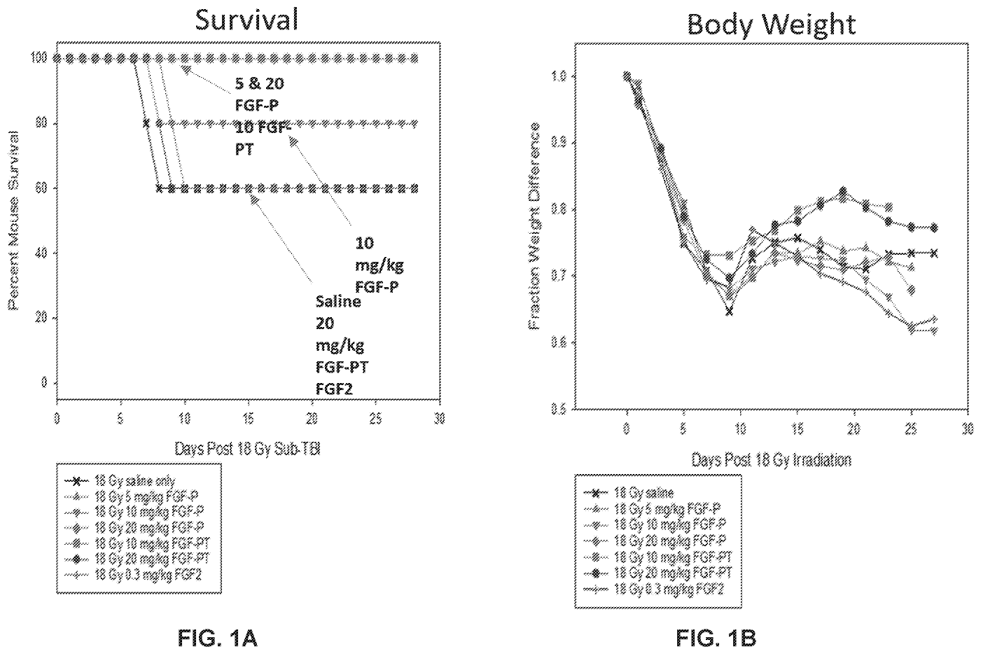

Improved survival and weight recovery at different doses of FGF-P and FGF-PT after irradiation at 18 Gy were observed (FIGS. 1A-1B).

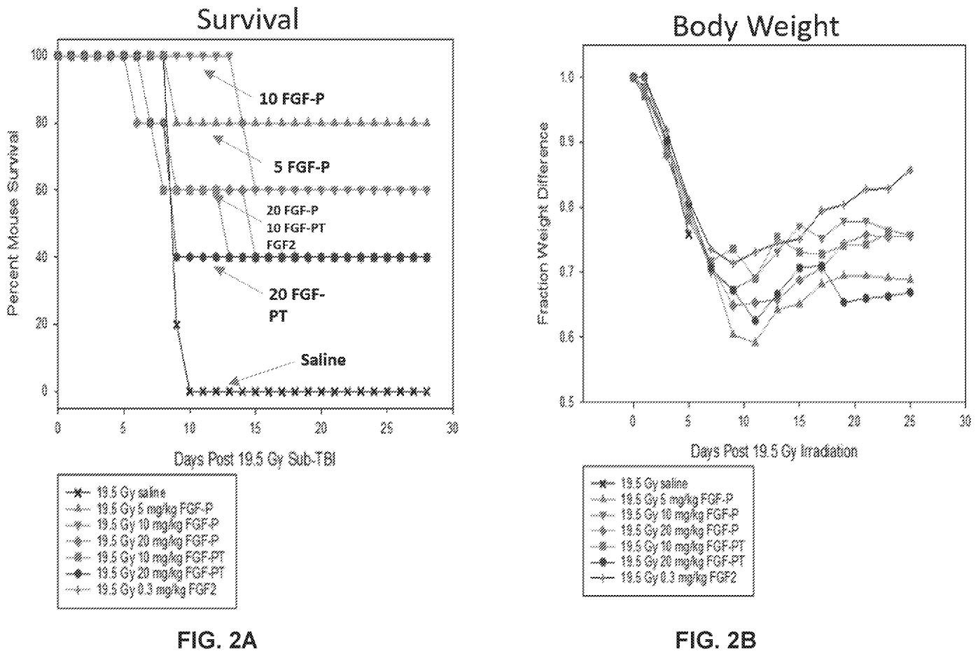

Improved survival and weight recovery at different doses of FGF-P and FGF-PT after irradiation at 19.5 Gy were also observed. Improved survival was observed at all FGF-P, FGF-PT, and FGF2 doses. Recovery was observed after severe weight loss (FIGS. 2A-2B).

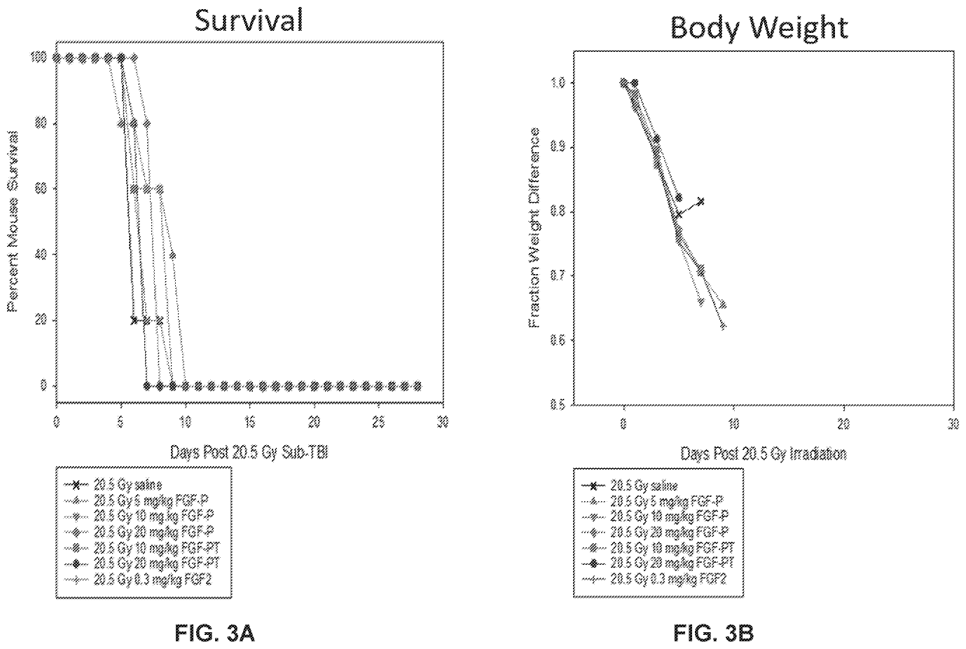

Improved survival and weight recovery at different doses of FGF-P and FGF-PT after irradiation at 20.5 Gy were observed. Improved survival was observed at many FGF-P, FGF-PT, and FGF2 doses, particularly, with FGF-P doses of 5-20 mg/kg with recovery after very severe weight loss (FIGS. 3A-3B).

### Example 2

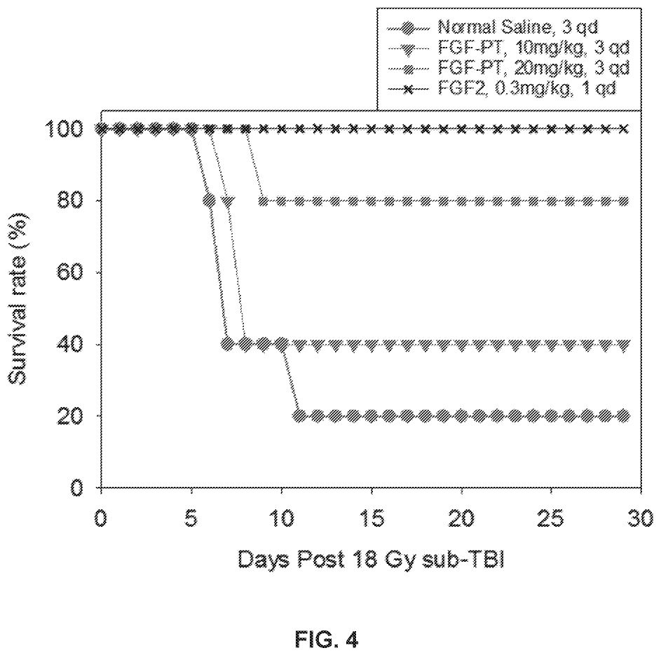

FIG. 4 shows survival rate (%) in 8-9 week old NIH Swiss male mice that have received a 18 Gy sub-TBI (one hind leg protected out of the radiation field) after subcutaneous doses of 10 and 20 mg/kg of FGF-PT administered once daily for three days beginning 24 hours post-irradiation, rhFGF2 administered at a dose of 0.3 mg/kg subcutaneously once at 24 hours post-irradiation, or vehicle control (normal saline) administered once daily for three days beginning 24 hours post irradiation.

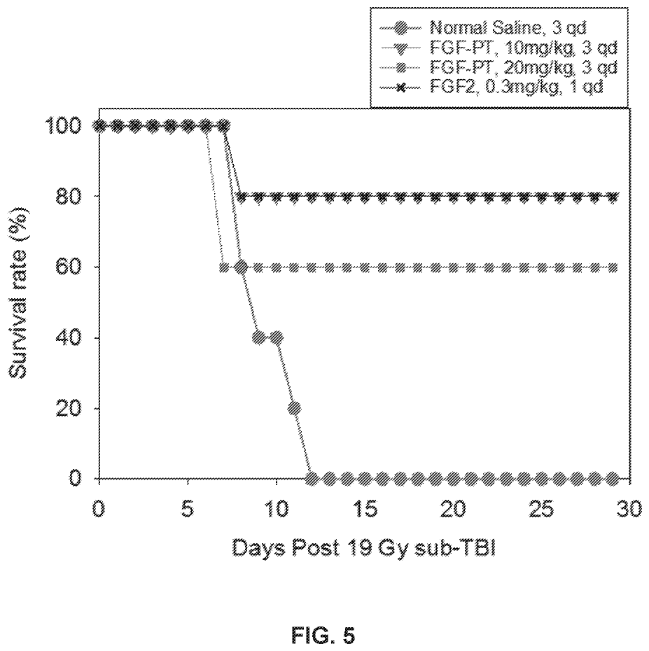

FIG. 5 shows survival rate (%) in 8-9 week old NIH Swiss male mice that have received a 19 Gy sub-TBI (one hind leg protected out of the radiation field) after subcutaneous doses of 10 and 20 mg/kg of FGF-PT administered once daily for three days beginning 24 hours post-irradiation, rhFGF2 administered at a dose of 0.3 mg/kg subcutaneously once at 24 hours post-irradiation, or vehicle control (normal saline) administered once daily for three days beginning 24 hours post irradiation.

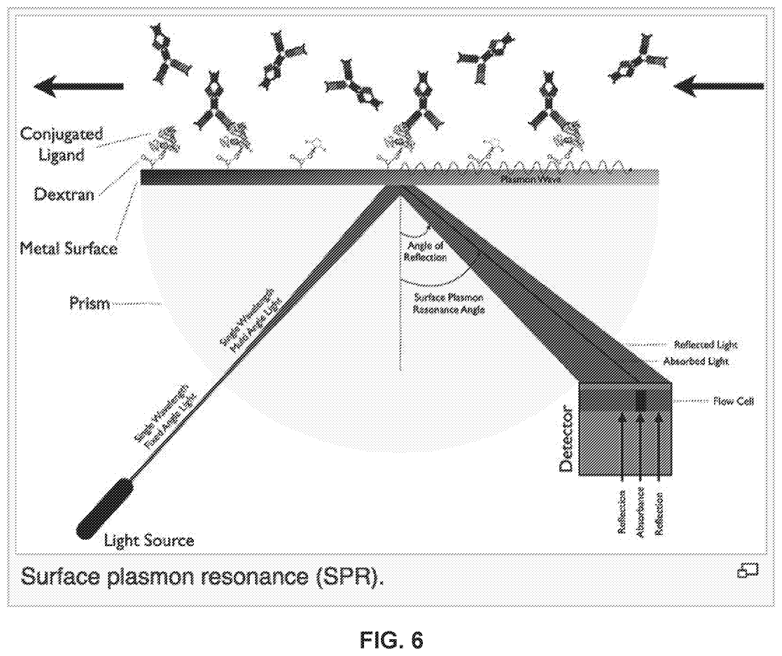

FIG. 6 illustrates a surface plasmon resonance method. In the SPR experiment, the ligands (eg. FGF receptors) are conjugated to the gold film surface of the reflective side of a prism. Single wavelength light is directed through the prism at a fixed angle relative to the reflective surface of the prism. The absorbance of the reflected light that corresponds to the surface plasmon resonance angle is measured while a run buffer containing an analyte (eg. FGF-PT) flows over the conjugated receptor ligand. The changes in the absorbance of the reflected light correspond to the extent of analyte binding to the conjugated ligand, producing binding kinetics plots that are then used to calculate the binding constants of the analyte/ligand binding event.

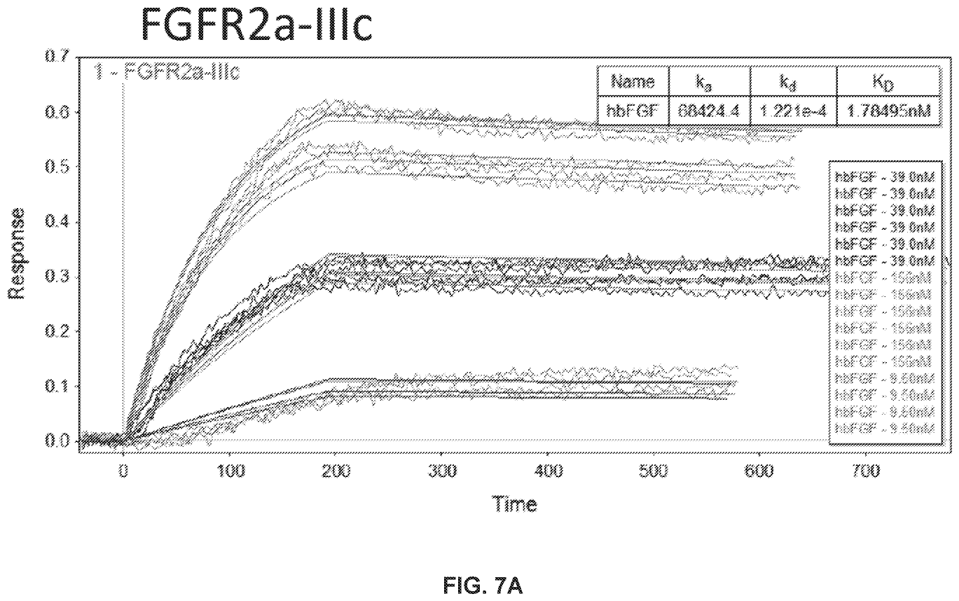

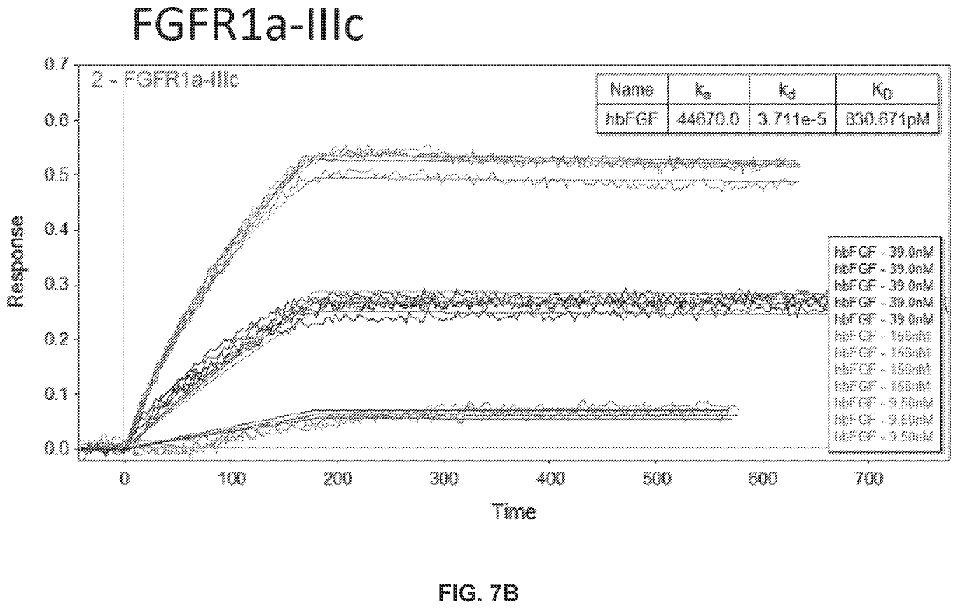

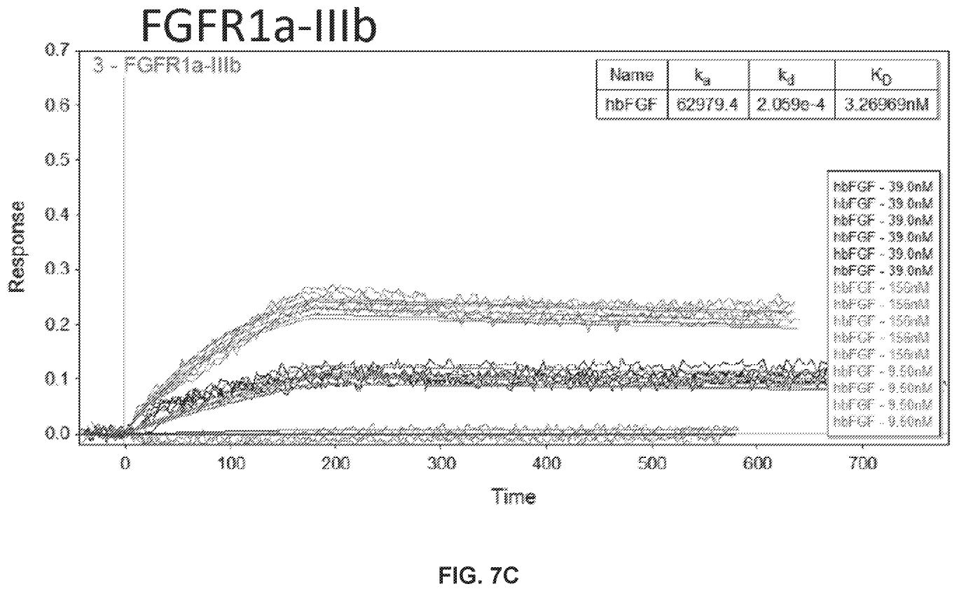

FIGS. 7A to 7C are fitted kinetic binding plots of hrFGF2 to the FGFR-2 alpha IIIc (FIG. 7A), FGFR-1 alpha IIIc (FIG. 7B), and FGFR-1 alpha IIIb (FIG. 7C) receptors. Five kinetic runs, one each for FGF-PT concentrations of 9.50, 39.0, 156 nM, are shown.

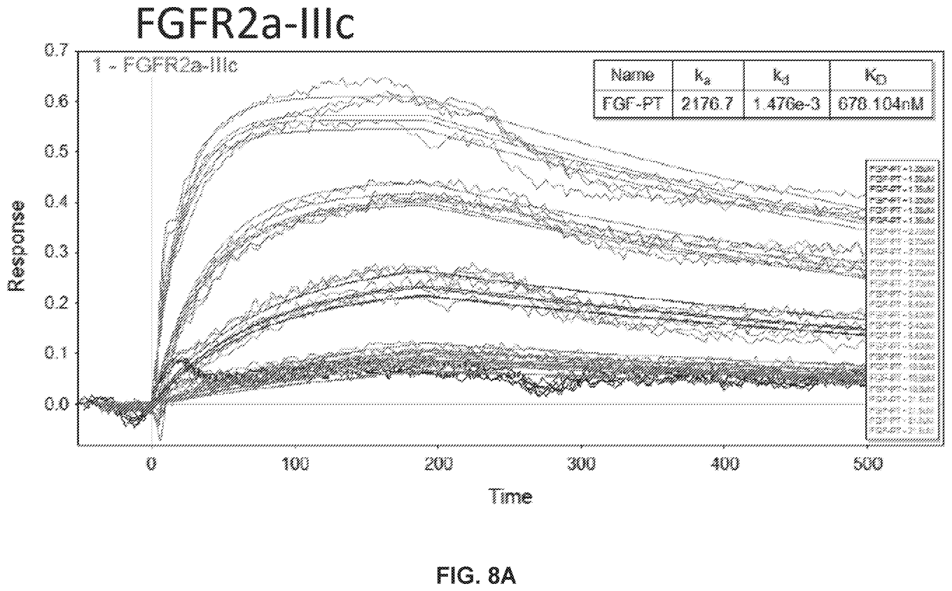

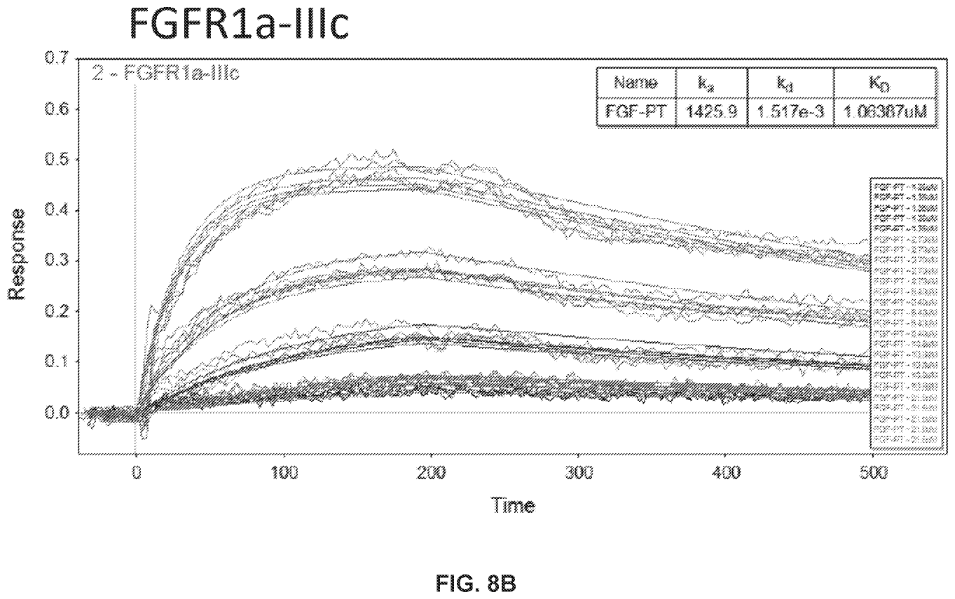

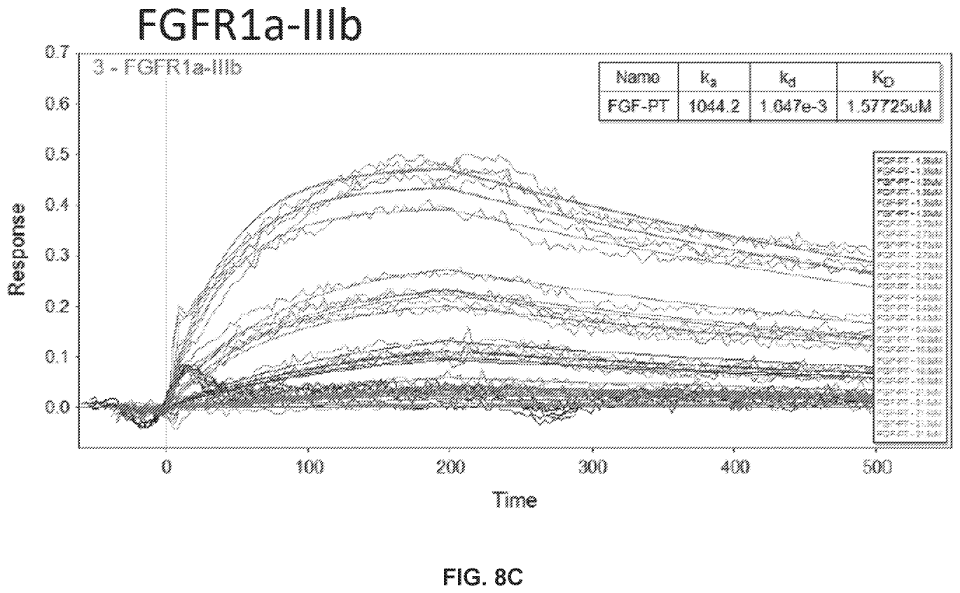

FIGS. 8A to 8C are kinetic binding plots of FGF-PT to the FGFR-2 alpha IIIc (FIG. 8A), FGFR-1 alpha IIIc (FIG. 8B), and FGFR-1 alpha IIIb (FIG. 8C) receptors. Five kinetic runs, one each for FGF-PT concentrations of 1.35, 2.70, 5.40, 10.8, and 21.6 uM, are shown.

Table 2 shows associative binding constants for recombinant human basic FGF (rhFGF2) and FGF-PT with three FGF receptor ligands. The constants were calculated from the binding kinetics of the rhFGF2 or FGF-PT analytes with the receptor ligands, as measured by the Horiba XelPlex surface plasmon resonance instrument. Receptors were immobilized on to the surface of the SPRi-Biochip using a standard concentration of 700 nM of receptor in the immobilization solution. Following receptor immobilization, differing concentrations of either the rhFGF2 or FGF-PT analytes in a run buffer are flowed over the receptors and the kinetics of analyte to ligand binding is followed over time. The binding constants are calculated from an analysis of the combined kinetic plots obtained from different analyte concentrations. The binding constants of FGF-PT for the three FGF receptors tested is approximately a 1000 fold higher than that found for rhFGF2. This compares fairly well to an approximate 100 fold difference in the doses of FGF-PT (10 mg/kg) and rhFGF2 (0.3 mg/kg) needed to gain survival benefit in the sub-TBI NIH Swiss model used to test efficacy of the agents in mitigating radiation toxic of the gastrointestinal track.

TABLE 2

| Receptor | rhFGF2 | FGF-PT |
|---|---|---|
| FGFR-1 alpha IIIb | 3.2 nM | 1.5 uM |
| FGFR-1 alpha IIIc | 830 pM | 1 uM |
| FGFR-2 alpha IIIc | 1.7 nM | 678 nM |

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A peptide having the sequence of SEQ ID NO: 2, provided that: a) one, two or, three amino acids of SEQ ID NO: 2 is substituted; b) an amino acid is added at the C-terminus and/or N-terminus of SEQ ID NO: 2; or c) one, two or, three amino acids of SEQ ID NO: 2 is substituted and an amino acid is added at the C-terminus and/or N-terminus of SEQ ID NO: 2.

2. The peptide of claim 1,
wherein X¹ of SEQ ID NO: 2 is substituted with D-tyrosine, tryptophan, phenylalanine, 4-amino phenylalanine, 4-chloro phenylalanine, 4-bromo phenylalanine, 4-iodo phenylalanine, 4-fluoro phenylalanine, 4-trifluoromethyl phenylalanine, 4-guanidino phenylalanine, 4-aminomethyl phenylalanine, 4-phosphonomethyl phenylalanine, 3,4-dichloro phenylalanine, 3,4-difluoro phenylalanine, 3,4-dihydroxy phenylalanine, 4-benzoyl phenylalanine, 4-bis(2-chloroethyl)amino phenylalanine, 3-nitro tyrosine, 3-chloro tyrosine, 3-amino tyrosine, 3-fluoro tyrosine or 2,6-dimethyl-tyrosines;
wherein X² of SEQ ID NO: 2 is substituted with D-arginine, lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid;
wherein X³ of SEQ ID NO: 2 is substituted with D-serine, threonine, alanine, 2-Amino-4-hydroxybutyric acid (homoserine), O-benzyl homoserine, O-methyl homoserine, 3-amino-2-hydroxypropanoic acid, O-phosphoserine, O-sulfoserine, O-nitroserine, β-(2-Thienyl)-serine, O-phosphothreonine, O-sulfothreonine, O-nitrothreonine;
wherein X⁴ of SEQ ID NO: 2 is substituted with D-arginine, lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid;
wherein X⁵ of SEQ ID NO: 2 is substituted with arginine, D-lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid;
wherein X⁶ of SEQ ID NO: 2 is substituted with D-tyrosine, tryptophan, phenylalanine, 4-amino phenylalanine, 4-chloro phenylalanine, 4-bromo phenylalanine, 4-iodo phenylalanine, 4-fluoro phenylalanine, 4-trifluoromethyl phenylalanine, 4-guanidino phenylalanine, 4-aminomethyl phenylalanine, 4-phosphonomethyl phenylalanine, 3,4-dichloro phenylalanine, 3,4-difluoro phenylalanine, 3,4-dihydroxy phenylalanine, 4-benzoyl phenylalanine, 4-bis(2-chloroethyl)amino phenylalanine, 3-nitro tyrosine, 3-chloro tyrosine, 3-amino tyrosine, 3-fluoro tyrosine or 2,6-dimethyl-tyrosine;
wherein X⁷ of SEQ ID NO: 2 is substituted with D-threonine, serine, β-hydroxy norvaline or O-phosphothreonine;
wherein X⁸ of SEQ ID NO: 2 is substituted with D-serine, threonine, alanine, 2-Amino-4-hydroxybutyric acid (homoserine), O-benzyl homoserine, O-methyl homoserine, 3-amino-2-hydroxypropanoic acid, O-phosphoserine, O-sulfoserine, O-nitroserine, β-(2-Thienyl)-serine, O-phosphothreonine, O-sulfothreonine, O-nitrothreonine;
wherein X⁹ of SEQ ID NO: 2 is substituted with D-tryptophan, tyrosine, phenylalanine, 1-methyl tryptophan, 5-fluoro tryptophan, 4-methyl tryptophan, 6-fluoro tryptophan, 7-azatryptophan, 5-benzyloxy tryptophan, 5-bromo tryptophan, 5-chloro tryptophan, 5-hydroxy tryptophan, 5-methoxy tryptophan, 6-chrloro tryptophan, 6-methyl tryptophan, 7-methyl tryptophan or 7-azatryptophan;
wherein X¹⁰ of SEQ ID NO: 2 is substituted with D-tyrosine, tryptophan, phenylalanine, 4-amino phenylalanine, 4-chloro phenylalanine, 4-bromo phenylalanine, 4-iodo phenylalanine, 4-fluoro phenylalanine, 4-trifluoromethyl phenylalanine, 4-guanidino phenylalanine, 4-aminomethyl phenylalanine, 4-phosphonomethyl phenylalanine, 3,4-dichloro phenylalanine, 3,4-difluoro phenylalanine, 3,4-dihydroxy phenylalanine, 4-benzoyl phenylalanine, 4-bis(2-chloroethyl)amino phenylalanine, 3-nitro tyrosine, 3-chloro tyrosine, 3-amino tyrosine, 3-fluoro tyrosine or 2,6-dimethyl-tyrosine;
wherein X¹¹ of SEQ ID NO: 2 is substituted with D-valine, isoleucine, leucine, β-cyclohexyl alanine, α,β-diamino propionic acid, propargylglycine, α-amino isobutyric acid, β-(2-pyridyl) alanine, β-(3-pyridyl) alanine, β-cyclopropyl alanine, β-t-butyl alanine, β-allyloxy carbonyl-α,β-diaminopropionic acid, 4-fluoro phenylglycine, 5,5,5-trifluoro leucine, 2-amino-3-(dimethylamino)-propionic acid or 2-amino caproic acid, allylglycine;
wherein X¹² of SEQ ID NO: 2 is substituted with D-alanine, serine, β-cyclohexyl alanine, α,β-diamino propionic acid, propargylglycine, α-amino isobutyric acid, β-(2-pyridyl) alanine, β-(3-pyridyl) alanine, β-cyclopropyl alanine, β-t-butyl alanine, β-allyloxy carbonyl-α,β-diaminopropionic acid, 4-fluoro phenylglycine, 5,5,5-trifluoro leucine, 2-amino-3-(dimethylamino)-propionic acid or 2-amino caproic acid, allylglycine;
wherein X¹³ of SEQ ID NO: 2 is substituted with D-leucine, isoleucine, valine, β-cyclohexyl alanine, α,β-diamino propionic acid, propargylglycine, α-amino isobutyric acid, β-(2-pyridyl) alanine, β-(3-pyridyl) alanine, β-cyclopropyl alanine, β-t-butyl alanine, β-allyloxy carbonyl-α,β-diaminopropionic acid, 4-fluoro phenylglycine, 5,5,5-trifluoro leucine, 2-amino-3-(dimethylamino)-propionic acid or 2-amino caproic acid, allylglycine;
wherein X¹⁴ of SEQ ID NO: 2 is substituted with arginine, D-lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid;
wherein X¹⁵ of SEQ ID NO: 2 is substituted with D-arginine, lysine, glutamine, monomethyl lysine, dimethyl lysine, trimethyl lysine, 5-hydroxylysine, hydroxylysine-5-sulfate, hydroxylysine-5-nitrate, hydroxylysine-5-phosphate, monomethyl arginine, asymmetric dimethyl arginine, symmetric dimethyl arginine, acetyl lysine, trifluoroacetyl lysine, palmitoyl lysine, citrulline, ornithine, diamino butyric acid, diamino propionic acid;
or any combination thereof.

3-16. (canceled)

17. The peptide of claim 1, wherein the substituted amino acid is in D-configuration.

18. The peptide of claim 1, wherein one amino acid from X¹ to X¹⁵ of SEQ ID NO: 2 is substituted.

19. The peptide of claim 1, wherein the peptide comprises the amino acid sequence SEQ ID NO: 5.

20. The peptide of claim 1, wherein the peptide is cyclized by forming an amide bond between X¹ to X¹⁵ of SEQ ID NO: 2.

21. The peptide of claim 1, wherein an amino acid is added at the C-terminus and/or N-terminus of SEQ ID NO: 2.

22. The peptide of claim 21, wherein the amino acid added to the C and the N terminus is cysteine, a combination of cysteine and an analog of cysteine, an analog of cysteine, or combination of analogs of cysteine that is capable of cyclizing the peptide through formation of a disulfide linkage.

23. The peptide of claim 21, wherein the amino acid added to the C and/or the N terminus is an analog of cysteine that is not capable of forming a disulfide linkage.

24. The peptide of claim 21, wherein the amino acid is added only to the C-terminus or only to the N-terminus of SEQ ID NO: 2.

25. The peptide of claim 21, wherein the amino acid is added to both the C and the N termini of SEQ ID NO: 2.

26. The peptide of claim 21, wherein a first amino acid is added to the C-terminus and a second amino acid is added to the N-terminus.

27. The peptide of claim 1, wherein an amino acid is added at the C-terminus and/or N-terminus of SEQ ID NO: 2 is cyclized through formation of an amide bond between the resulting C-terminus and N-terminus.

28. A salt of the peptide of claim 1, wherein the salt is with: hydrochloric acid, hydrobromic acid, perchloric acid, nitric acid, thiocyanic acid, sulfuric acid, phosphoric acid, trifluoroacetic acid (TFA), formic acid, acetic acid, propionic acid, hexanoic acid, cyclopentanepropionic acid, glycolic acid, lactic acid, pyruvic acid, oxalic acid, malic acid, malonic acid, succinic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-di sulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, sodium hydroxide, ammonium hydroxide, potassium hydroxide, mono-, di-, trialkyl and aryl amines, ethanolamines, diethanolamine, triethanolamine, N-methylglucamine, sodium, potassium, calcium, magnesium, ammonium, tetraalkylammonium.

29. (canceled)

30. A composition comprising the peptide of claim 1 and a pharmaceutically acceptable carrier and/or an excipient.

31. A method of treating a disease in a subject, comprising administering to the subject the composition of claim 30, wherein the disease affects rapidly proliferating tissue, or wherein the disease occurs due to radiation exposure.

32-33. (canceled)

34. The method of claim 31, wherein the disease is alimentary mucositis, oral mucositis, gastrointestinal mucositis, disease of hematopoiesis, anemia, leukopenia, thrombocytopenia, pancytopenia, clotting disease, bone marrow failure, graft-versus-host disease, radiation induced prostatitis, vaginitis, urethritis or a cardiovascular/central nervous system syndrome, diarrhea, skin burn, sores, fatigue, dehydration, inflammation, hair loss, ulceration of alimentary tract mucosa, xerostomia, bleeding, aneurysm or a combination thereof.

35. The method of claim 31 comprising administering the peptide to the subject: before exposed to radiation, after exposure to radiation and prior to the development of the disease or after exposure to radiation and after the development of the disease.

36. The method of claim 31, comprising inducing angiogenesis in a tissue in the subject by administering the peptide to the tissue, wherein inducing angiogenesis in the tissue treats the disease.

37. (canceled)

38. The method of claim 31, comprising stimulating growth and proliferation of cells in a tissue in the subject by administering the peptide to the tissue, wherein inducing growth and proliferation of cells treats the disease.

39. (canceled)


SEQUENCE LISTING (5 sequences)

SEQ ID NO: 1 - protein, 15 aa, Mus musculus
YRSRKYSSWYVALKR

SEQ ID NO: 2 - protein, 15 aa, Homo sapiens
YRSRKYTSWYVALKR

SEQ ID NO: 3 - protein, 17 aa, Artificial Sequence
  Synthetic Construct
CYRSRKYSSWYVALKRC

SEQ ID NO: 4 - protein, 17 aa, Artificial Sequence
  Synthetic Construct
CYRSRKYTSWYVALKRC

SEQ ID NO: 5 - protein, 15 aa, Artificial Sequence
  Synthetic Construct
  MISC_FEATURE (1)..(1): Xaa is a tyrosine or a tyrosine analog
  MISC_FEATURE (2)..(2): Xaa is a arginine or an arginiine analog
  MISC_FEATURE (3)..(3): Xaa is a serine or a serine analog
  MISC_FEATURE (4)..(4): Xaa is a arginine or an arginiine analog
  MISC_FEATURE (5)..(5): Xaa is a lysine or an lysine analog
  MISC_FEATURE (6)..(6): Xaa is a tyrosine or a tyrosine analog
  MISC_FEATURE (7)..(7): Xaa is a threonine or a threonine analog
  MISC_FEATURE (8)..(8): Xaa is a serine or a serine analog
  MISC_FEATURE (9)..(9): Xaa is a tryptophan oir a tryptophan analog
  MISC_FEATURE (10)..(10): Xaa is a tyrosine or a tyrosine analog
  MISC_FEATURE (11)..(11): Xaa is a valine or a valine analog
  MISC_FEATURE (12)..(12): Xaa is a alanine or a alanine analog
  MISC_FEATURE (13)..(13): Xaa is a leucine or a leucine analog
  misc_feature (14)..(14): Xaa can be any naturally occurring amino acid
  MISC_FEATURE (15)..(15): Xaa is a arginine or an arginine analog
XXXXXXXXXXXXXXX